(19) United States
(12) Patent Application Publication
(10) Pub. No.: US 2009/0130704 A1
(43) Pub. Date: May 21, 2009

(54) Novel bioreactor

(76) Inventor: Dale C. Gyure, Belmont, MA (US)

Correspondence Address: GREENLEE WINNER AND SULLIVAN P C, 4875 PEARL EAST CIRCLE, SUITE 200, BOULDER, CO, 80301, US

(21) Appl. No.: 10/987,954

(22) Filed: Nov. 12, 2004

Related U.S. Application Data
(60) Provisional application No. 60/520,386, filed on Nov. 13, 2003.

(51) Int. Cl. C12P 1/00; C12M 1/04

(52) U.S. Cl. 435/41; 435/292.1

(57) ABSTRACT

This invention provides bioreactors having a selectively permeable porous material with an open pore structure, useful for producing products including hydrogen gas, biomass, chemicals, and pharmaceuticals. The porous materials are utilized, for example, as one or more portions of or entire walls, covers, floors, filters, windows, or tubes of the bioreactors. This invention provides bioreactors comprising porous materials that are aerogels, xerogels, or sol-gel glasses, including silica aerogels. The selectively permeable porous materials are gas-permeable, and in addition optionally photopermeable, transparent, hydrophobic, and/or capable of functioning as sterile barriers. This invention provides methods for culturing cells and organisms employing the bioreactors of the invention. This invention further provides methods for producing gaseous products, including hydrogen, biomass, chemicals, and pharmaceuticals employing the bioreactors of the invention.

### CROSS-REFERENCE TO RELATED APPLICATIONS

This application takes priority under 35 U.S.C. 119(e) to U.S. Provisional Patent Application Ser. No. 60/520,386, filed Nov. 13, 2003, which is incorporated by reference in its entirety herein.

### FIELD OF THE INVENTION

The subject matter of this invention relates to bioreactors useful for producing products including hydrogen and other gaseous products, biomass, chemicals, and pharmaceuticals; and selectively permeable materials having an open pore structure useful as structural and functional components in bioreactors.

### BACKGROUND OF THE INVENTION

Methods for making porous materials having an open pore structure having one or more properties selected from: gas permeability, substantially non-selective gas permeability, two-way gas permeability, gas permeability by methods other than molecular diffusion, photopermeability, pores having a diameter between about 0.1 nanometer to about 1000 nanometers, hydrophobicity, ability to be made in controlled shapes, ability to exclude and/or contain a microorganism and function as a sterile barrier, and the ability to be recycled; are known in the art, and include use of a sol-gel process (U.S. Pat. No. 6,440,711; Akimov (2003) Instruments and Experimental Techniques 46:287-299; and U.S. Pat. No. 6,303,290, issued Oct. 16, 2001). In sol-gel technology, simple molecules, monomers or precursors, are suspended in solution and reacted or polymerized with each other to form a sol or a collection of colloidal clusters. The macromolecules become bonded and cross-linked, forming a gel called a sol-gel. Sol-gel glasses, aerogels, and xerogels are produced by carefully drying the sol-gel to remove solvents so that the fragile gel network does not collapse. Many methods are known in the art for drying sol-gels (Sol-Gel Synthesis and Processing (Ceramic Transactions, Vol. 95), Sridhar Komarneni, Sumio Sakka, Pradeep P. Phul, Richard M. Laine, The American Ceramic Society, May, 1999 and Sol-Gel Science and Technology: Topics in Fundamental Research and Applications, Sumio Sakka, Kluwer Academic Publishers, November, 2002). Porous materials made using a sol-gel process using certain precursors can be made to have an open pore structure, to be gas permeable, and/or to be photopermeable. Open pore structured porous materials can be made to a selected degree of hydrophobicity by methods including use of precursors with hydrophobic moieties or surface modifiers having hydrophobic moieties.

Aerogels can have the highest internal surface area per gram of material of any known material. They can exhibit the best electrical, thermal, and sound insulation properties of any known solid. They can be made photopermeable and/or transparent. The structure and density of the final aerogel product are influenced by precursors or monomers and reaction conditions such as temperature, pH, types of catalysts, choice of solvent, method of drying etc.

A variety of aerogel compositions are known in the art. Many are based on silica (silicon dioxide). However, other materials such as metal oxides, plastics (such as polyurethane), and natural and synthetic rubbers can be used to make aerogels.

Methods of making aerogels, xerogels and sol-gel glasses are described in “Sol Gel Processing of Ceramics and Glass,” Market Report, July 2002, Business Communications Co, Norwalk, Conn., USA. U.S. Pat. No. 5,851,947, issued Dec. 22, 1998, describes aerogels containing atomically dispersed noble metals. U.S. Pat. No. 5,624,875, issued Apr. 29, 1997, describes an inorganic porous material and process for making same.

Aerogels, xerogels, and sol-gel glasses have been used to make specialty glasses, advanced optical coatings, advanced ceramics, flat-panel displays, batteries, capacitors, refrigeration, insulation, and catalysts (“Aerogels and Xerogels: Growth and Opportunities for the Early 21^{st} Century,” Market Report, 1996, Technical Insights, John Wiley and Sons, Inc., San Antonio, Tex., U.S.A.). Akimov (2003) Instruments and Experimental Techniques 46:287-299 is a review article describing applications of aerogels. Jensen (1992) Journal of Non-Crystalline Solids 145:237-239 describes the use of an aerogel as a transparent insulation material between two plates of glass, for use in windows, solar walls, and sunspaces.

Various materials have been used to embed, encapsulate, or provide a support network for biological components. U.S. Pat. No. 5,895,757, issued Apr. 20, 1999, and U.S. Pat. No. 5,693,513, issued Dec. 2, 1997, describe encapsulation of living tissue cells in inorganic microspheres prepared from an organosilicon. U.S. Pat. No. 5,624,875, issued Apr. 29, 1997, describes a process for producing inorganic materials that are reported to be useful as columns that can carry enzymes, such as in a (bio)reactor. The inorganic materials are not described as useful as walls, covers, filters or other structural and/or functional components in (bio)reactors other than as support networks for biological components, e.g., for carrying enzymes, catalysts, or (bio)sensor components. U.S. Pat. No. 6,303,290, issued Oct. 16, 2001, describes encapsulation of biomaterials in porous glass-like matrices prepared via an aqueous colloidal sol-gel process.

U.S. Pat. No. 5,069,794 (issued Dec. 3, 1991) describes a method for separating mixture components using non-composited microporous membranes comprising a continuous array of crystalline molecular sieve material (zeolitic membranes). Zeolites are microporous crystalline solids with well-defined structures. Generally they contain silicon, aluminum, and oxygen in their framework and migrating cations, water and/or other molecules within the spaces in the crystal lattice. Many occur naturally as minerals, and are extensively mined in many parts of the world. Others are synthetic, and are made commercially for specific uses. Zeolites comprise a framework based on an infinitely extending three-dimensional network of SiO₄ and [AlO₄]⁻¹ tetrahedra linked though common oxygen atoms. The framework structure encloses cavities which are empty or occupied by large ions and water molecules, all of which have considerable freedom of movement, permitting ion exchange and reversible dehydration. Zeolites typically have pore sizes of about 3 angstroms to about 12 angstroms (0.3 to 1.2 nanometers). U.S. Pat. No. 5,464,798 (issued Nov. 7, 1995) describes a zeolite composite membrane and use of the membrane for separating vapor/gas mixtures. U.S. Pat. No. 6,503,295 (issued Jan. 7, 2003) describes a method for separating gas components of a feedstream using mixed matrix membranes. U.S. Pat. No. 5,403,799, issued Apr. 4, 1995, describes a macroporous, upset-resistant inorganic oxide catalyst support comprising a zeolite particle. Zeolites do not have an open pore structure, but instead have atomic-sized spaces within a regular crystal lattice. Zeolites have been utilized to absorb gases. Absorbance occurs when a gas molecule interacts with the electrical fields generated by the stationary atoms that are a part of the zeolite crystal structure. Over time, the gas permeability of a zeolite can decrease as smaller openings become filled with gas molecules. Zeolites have also been utilized to separate gases by steric exclusion. Zeolites are fabricated by a crystallization process. They cannot be fabricated by a sol-gel process. Zeolites are not photopermeable.

Ceramic membranes are useful as separation devices (Recent advances in gas separation by microporous ceramic membranes, edited by N. K. Kanellopoulos, New York, Elsevier, 2000). Ceramic membranes are not actually membranes, but separation devices. Ceramic membranes are made by a sintering process, not a sol-gel process, and ceramic membranes are not photopermeable. They can be made using a ceramic tube that is cast and fired using traditional methods for making ceramic products. Progressively smaller ceramic particles are then layered onto the inside surface of the tube and it is fired again. Each subsequent layer is made of finer and finer particles. The outermost layer on the inner surface of the tube determines the size rating of the filter. The size is determined by the interstitial spaces between the ceramic particles that have been fired into place. The tubes are then potted within a tube having a greater diameter to make the separation device. Ceramic membrane tubes are very brittle requiring that they be operated vertically to prevent their own weight, in the horizontal position, from causing them to sag and crack. Ceramic membranes are made from gamma Al₂O₃ and alpha Al₂O₃ as well as ZrO₂. Both the alumina oxides are resistant to acids, but do gradually dissolve at high pH, hence they are not ideal for applications where high pH solutions are used, e.g., for cleaning bioreactors.

There is a need in the art for efficient and economical bioreactors capable of culturing cells, organisms, and cellular components, that are gas permeable, photopermeable, capable of maintaining monoseptic conditions, and scalable to industrial production size. There is a need in the art for bioreactor materials that are gas permeable, photopermeable, scalable, able to be made in a wide variety of forms, able to function as sterile barriers, resistant to chemical attack, and/or recyclable.

Many bioreactor designs are known in the art. Bioreactors can be categorized into five classes by methods utilized for gas exchange, presence, or absence of means for photoradiation delivery, and ability to maintain monoseptic conditions.

TABLE 1
Bioreactor Classes

| | Gas | Separate | Photora- | Mono- | |
|---|---|---|---|---|---|
| | Introduction | Gas Exit | diation | septic | Agitation |
| Class I | forcibly | yes | no | yes | yes |
| | introduced as | | | | |
| | separate phase | | | | |
| Class II | passive, not | optional | no | yes | optional |
| | distinct phase | | | | |
| Class III | forcibly | yes | yes | yes | yes |
| | introduced as | | | | |
| | separate phase | | | | |
| Class IV | passive, not | optional | yes | yes | optional |
| | distinct phase | | | | |
| Class V | passive | no | optional | no | optional |

Class I bioreactors have organisms or cells contained and isolated physically from the outside environment to maintain monoseptic conditions within the bioreactor. Gases are forcibly introduced and/or injected as a distinct phase into the culture fluid. Gases include those used for respiration, aerobic metabolism as well as inert gases used to promote anaerobic conditions and sweep gaseous and volatile products and by-products out of the reactor. Universally, in this class of reactors, product and by-product gases exit the reactor via a means separate and independent from the point of forced introduction. The culture fluid is forcibly agitated (i.e., hydraulic movement by means other than natural convection) either by a separate stirrer or by the forced introduction of gases as a distinct phase into the culture fluid. Examples include conventional stirred tank and airlift bioreactors for the production of chemicals, pharmaceuticals, and small molecules.

Class II bioreactors have organisms or cells contained and isolated physically from the outside environment to maintain monoseptic conditions within the bioreactor. Gases are passively introduced to the culture fluid, but NOT as a distinct phase. Means of introduction or transfer can be by migration through a gas-permeable material that separates the bulk gas phase from the bulk liquid phase and also serves to isolate the culture fluid physically from the outside environment. Gases include those used for respiration, aerobic metabolism as well as inert gases used to promote anaerobic or other special conditions within the reactor. Product and/or by-product gases exit the bioreactor via a separate vent as a distinct phase or via migration through the same or another section of a gas permeable material that also serves to isolate the culture fluid physically from the outside environment. The culture fluid may or may not be forcibly agitated. Examples include small and large cell culture reactors and microbial reactors for the production of chemicals, pharmaceuticals, or gases.

Class III bioreactors are identical to Class I bioreactors except that the culture fluid is illuminated or photoradiated to provide electromagnetic radiation as an integral raw material or component of the process. Variations are known in the art for maximizing light delivery to the organisms (e.g., Gordon (2002) Intl J of Hydrogen Energy 27:1175-1184). Examples include conventional bioreactors that are also illuminated either internally or externally for the production of chemicals, pharmaceuticals, or small molecules. Examples include bioreactors whose purpose includes deactivation of all organisms or cells by exposure to electromagnetic radiation.

Class IV bioreactors are identical to Class II bioreactors except that the culture fluid is illuminated or photoradiated to provide electromagnetic radiation as an integral raw material or component of the process. Examples include small and large cell culture reactors and microbial reactors for the production of chemicals, pharmaceuticals, or gases. Examples include bioreactors whose purpose includes deactivation of all organisms or cells by exposure to electromagnetic radiation.

Class V bioreactors have organisms or cells that are not isolated physically from the outside environment. Monoseptic conditions are not maintained within the bioreactor. Gases are passively introduced to the culture fluid by mass transfer from a bulk gas phase present above and in contact with the surface of the culture fluid. Mass transfer occurs passively at the gas liquid interface. Mass transfer is passive because the bulk gas phase is not pumped or injected. Gases include those used for respiration, aerobic metabolism as well as inert gases used to promote anaerobic or other special conditions within the culture fluid. Product and/or by-product gases leave the culture fluid via the same gas-liquid interfacial area. The culture fluid may or may not be forcibly agitated. The culture fluid may or may not be photoradiated. Examples include open ponds for cultivation of photosynthetic algal biomass (can be used as a dietary supplement) or for the production of chemicals using photosynthetic algae and/or photosynthetic bacteria as well as aerobic bacteria. Examples also include aerobic digestion of soluble organic matter by mixed bacterial cultures in a wastewater treatment plant. In Class V bioreactors selectively porous materials function as a barrier to minimize contamination of the culture fluid with foreign debris, while permitting photoradiation and gas exchange, if necessary.

Additional classes of bioreactors exist having different combinations of methods for gas exchange, presence or absence of means for photoradiation delivery, and ability to maintain monoseptic conditions.

Bioreactor designs, instructions for constructing bioreactors, and methods for using bioreactors can be found in: Bioreactor Design Fundamentals by Norton G. McDuffie, October '91, 137 pp. Pub: Butterworth-Heinemann. ISBN 0750691077; Bioreactor System Design by Juan A. Asenjo and J. C. Merchuk, January '95, 648 pp. Pub: Mercel Dekker. ISBN 0824790022; Bioreactors in Biotechnology by A. H. Scragg, September. '91, 300 pp, Pub: Prentice Hall Professional Technical References. ISBN 0130851434; Cell Culture Systems and Conventional Bioreactor Technology by H. Michelle Jones, November '97, 141 pp. Pub: Business Communications. ISBN 1569653828; Growth and Synthesis: Fermenters, Bioreactors and Biomolecular Synthesizers by William L. Hochfeld, October '94, 266 pp. Pub: CRC Press. ISBN 0935184627; Membrane Bioreactors: Feasibility and Use in Water Reclamation by Samer Adham and R. Shane Trussell, January '01, Pub: Water Environment Research Foundation. ISBN 1893664368; Operational Models of Bioreactors by Biotol Partners Staff, July '92, 282 pp. Pub: Butterworth-Heinemann. ISBN 0750615087; 3^{rd} International Conference on Bioreactor and Bioprocess Fluid Dynamics by American Society of Mechanical Engineers Staff, 568 pp. Pub: Professional Engineering Publishing; Advances in Biochemical Engineering—Biotechnology: Bioreactor Systems and Effects by A. Fiechter (ed.), October '91, 156 pp. Pub: Springer-Verlag New York, Inc. ISBN 0387540946; Fermentation & Bioreactors, August '87, Pub: Business Communications. ISBN 0893364045; Bioreaction Engineering Principles by Jens H. Nielsen, July '94, 480 pp. Pub: Kluwer Academic Publications ISBN 030644688X; Airlift Bioreactors by M. Y. Chisti, January '89, 350 pp. Pub Elsevier Science ISBN 1851663207; Animal Cell Bioreactors by Chester S. Ho (ed.) and Daniel I. Wang (ed.), January '91, 512 pp. Pub: Butterworth-Heinemann ISBN 0409901237; Basic Bioreactor Design by Klaas Van Riet and J. Tramper, January '91, 480 pp. Pub: Marcel Dekker ISBN 0824784464; Bioreactor Design and Product Yield by Biotol Board Staff, August '92, 275 pp. Pub: Butterworth-Heinemann ISBN 0750615095; On-line Estimation and Adaptive Control of Bioreactors by G. Bastin and D. Dochain (ed.), January '90 ISBN 0444884300; Bioreaction Engineering, Vol. 2 Characteristic Features of Bioreactors by K. Schugerl, May '91, 418 pp. Pub: John Wiley & Sons ISBN 0471925934; Membrane Systems Analysis and Design: Applications in Biotechnology, Biomedicine and Polymer Science by W. R. Vieth, December '88, 360 pp. Pub: John Wiley & Sons; BioCatalytic Membrane Reactors by Enrico Drioli and Lidietta Giorno, February '99, 211 pp. Pub: Taylor & Francis, Inc. ISBN 0748406549; Biological Reaction Engineering: Principles, Applications and Modeling with PC Simulation by I. J. Dunn, J. Ingham, E. Heinzle and J. E. Prenosil, November '92, 438 pp. Pub: John Wiley & Sons ISBN 3527285113; Multiphase Bioreactor Design by Joaquim M. Cabral (ed.), J. Tramper (ed.) and Manuel Mota (ed.), December '01, 528 pp. Pub: Taylor & Francis, Inc. ISBN 0415272092; and Bioreaction Engineering: Modeling and Control by K. Schugerl and Karl-Heinz Bellgardt (ed.), January '00, 600 pp, Pub: Springer-Verlag New York, Inc. ISBN 354066906X.

Class I, II, III, and IV bioreactors can be batch, semi-batch or continuous systems and the geometry of any of the Classes I through IV can be a cylindrical vessel, a box shape, a tubular shape, etc. The open pond (Class V) style occurs in a variety of configurations. One example, called a racetrack design has a circulation means (e.g., a paddle slowly circulates the culture fluid) around a very long and narrow donut shaped bathtub that is open to the atmosphere. Other designs include a simple stagnant pond and an agitated bathtub where there is no pathwise movement of the culture fluid.

Parameters to be considered when selecting a bioreactor class include availability of and requirements for photoradiation sources, photobioreactor construction materials, availability of gases, means for gas exchange, regional climate (Class V), land availability (Class V), requirements for gas exchange (e.g., highly aerobic systems with high oxygen demand require more exchange than is typically provided by passive diffusion), shear sensitivity of the cell, organism, or cellular component to be cultured, whether the cells need to anchor themselves on the wall of the bioreactor (mammalian cells may need to be anchored to something in order to grow), and what type of product will be made, e.g., gaseous product, soluble product or insoluble product.

Photobioreactors can optionally include means for monitoring and controlling temperature, circulation and/or flow rates, and pH; monitoring and/or delivering dissolved gases including oxygen, nitrogen, sulfur dioxide, carbon dioxide, hydrogen sulfide, and hydrogen; monitoring and/or delivering nutrients; monitoring culture dilution; providing an atmospheric seal; delivering and detecting photoradiation; optimizing photoradiation delivery, e.g., reorienting a portion of the bioreactor relative to a source of photoradiation; biomass mixing; prevention of settling; removal of liquid, solid, and gaseous wastes; removal of gases interfering with selected biochemical pathways such as dissolved oxygen and carbon dioxide gases; depletion of sulfur; protection of contamination from unwanted particles, including microorganisms (e.g., sterility); sanitizing and/or sterilizing the bioreactor; harvesting gaseous products, e.g., hydrogen; and harvesting non-gaseous products. Automatic monitoring and/or delivery can be performed using a computer.

Bioreactors useful for hydrogen production include a means for harvesting the gas product, hydrogen. All five classes of bioreactors are useful for producing hydrogen. Means are known in the art for selecting an appropriate bioreactor style for the organism and biochemical pathway(s) selected for utilization for hydrogen production.

Cyanobacterial photobioreactors (PBRs) for hydrogen production include glasstube photobioreactors, membrane photobioreactors, and spiral tubular bioreactors. Glasstube PBRs have a light-receiving glasstube part that is not gas permeable. Membrane photobioreactors have a membrane for separating cells from the H₂ stream. Both media and dissolved hydrogen pass through the membrane, wasting large amounts of media for the amount of hydrogen produced. Membranes can be in hollow-fiber, or flat or spiral sheet forms. Membranes have been made from a variety of materials including cellulose acetate and nitrate, polyvinylidene difluoride, polysulfone, polypropylene, polytetrafluoroethylene, cuprammonium rayon, and polyacrylonitrile, (BioHydrogen Chapter 47, Markov), none of which are photopermeable. Polyvinyl chloride has also been utilized as a membrane material. These membranes are liquid permeable, to contain the cells and allow liquids containing dissolved gases to pass through. These membranes are not used to completely contain the liquid culture suspending the cells and are not photopermeable.

BioHydrogen (1998) Plenum Press, NY, ed. Zaborsky, is a compilation of talks given at the Proceedings of an International Conference on Biological Hydrogen Production in 1997. Chapter 40 (Ikuta et al.) describes hydrogen by photosynthetic microorganisms using bioreactors made with acrylic resin or polyvinyl chloride. Chapter 41 (Ogbonna et al.) describes large scale photobioreactors made with glass. Chapter 43 (El-Shishtawy et al.) describes a photobioreactor having a polyacrylate light-receiving face and a modified polyester diffusion/reflection sheet. Chapter 45 (Otsuki et al.) describes a floating type bioreactor made with an acrylic plate. Chapter 47 (Markov) is described above. Chapter 48 (Tredici et al.) describes a cost comparison of various bioreactors for hydrogen production.

Hoekema (2002) Intl J of Hydrogen Energy 27:1331-1338 describes a pneumatically agitated flat-panel photobioreactor for photoheterotrophic hydrogen production using Rhodopseudomonoas sp. A stainless-steel frame separated three polycarbonate panels into two compartments. A membrane pump was used to circulate gas (air) through spargers (hypodermic needles). Hoekema suggests the use of argon gas with photoheterotrophs.

U.S. Pat. No. 5,763,279 (issued Jun. 9, 1998) describes a gas permeable bioreactor made at least partially of gas permeable materials, such as silicone rubber, polytetrafluoroethylene, polyethylene, mixtures of silicone rubber with other plastics, and silicone rubber coated cloth, which are gas permeable by molecular diffusion, as well as porous polytetrafluoroethylene, which is a foamed plastic not having an open pore structure.

U.S. Pat. No. 6,228,607 (issued May 8, 2001) describes a bioreactor comprising a liquid permeable membrane and a gas permeable membrane for allowing the passage of oxygen. No useful examples of gas permeable membranes are reported.

U.S. Pat. No. 5,137,828 (issued Aug. 11, 1992) describes a biomass production apparatus comprising a substantially transparent tube made of polyethylene wound on an upstanding core structure.

U.S. Pat. No. 6,395,521 (issued May 28, 2002) describes a microbial process for producing hydrogen using a transparent tower-type air-lift culture tank. Transparent materials useful for making the culture tank include transparent plastics such as acrylic resin, polycarbonate, polypropylene, polyterephthalate, and glass, which are not considered gas permeable.

U.S. Pat. No. 6,432,698 (issued Aug. 13, 2002) describes a disposable bioreactor for culturing microorganisms and cells, such as members of the genus Caenorhabditis. Preferred materials for constructing the bioreactor include flexible or semi-flexible water proof sheets, such as plastic, sealed along their edges to form a container. The container also comprises inlet and outlet ports for introducing and exhausting or removing liquids and/or gases.

WO 02/31101 (filed on Oct. 10, 2001) describes a plastic, sterilizable bioreactor.

WO 96/21723 (filed on Dec. 20, 1995) describes an apparatus for biomass production having a substantially transparent chamber made of a material such as flexible polyethylene or polyvinyl chloride.

EP 0 100 660 (filed on Jul. 29, 1983) describes bioreactors comprising a glass-like gel comprising a metal hydroxy compound as a carrier for immobilizing peptide-containing compounds, formed using a material such as an alkoxy silane.

UK Patent application GB 2118572 describes a photobioreactor comprising a flow part constructed at least in part of a transparent material capable of passing illumination, such as glass, polyvinyl chloride, or other resinous material.

JP 6000494 (published Jan. 11, 1994) describes a bioreactor having fibers or fibrous gel in the vicinity of air holes.

JP 9051794 (published Feb. 25, 1997) describes a polyurethane gel, swelled with water, having communicating pores and high strength, capable of immobilizing cells.

Published EP application 0413027 published February 1991 (and corresponding published PCT application WO90/01538 (February 1990)) relate to bioreactors using open-cell porous ceramic carriers. An open-cell porous ceramic carrier exemplified as “cordierite (2MgO.2Al₂O₃.5SiO₂)+alumina (Al₂O₃)” is reported useful as a carrier or matrix for adhering and immobilizing a biocatalyst (e.g., cells).

Trickle bed bioreactors (TBRs) are known in the art (Wolfrum, Proceedings of the 2002 US DOE Hydrogen Program Review, NREL/CP-610-32405, “Bioreactor Development for Biological Hydrogen Production”; Wolfrum, Proceedings of the 2001 US DOE Hydrogen Program Review, NREL/CP-570-30535; Wolfrum (2002) Applied Biotechnology and Bioengineering 98-100:611-625, “Bioreactor Design Studies for a Novel Hydrogen-Producing Bacterium”).

Liang et al. (2002) Intl J of Hydrogen Energy 27:1157-165 describes a fermentation reactor which uses a silicone rubber membrane to separate carbon dioxide from the organisms by molecular diffusion, when the concentration of dissolved CO₂ in the medium is more than that in equilibrium with the partial pressure of CO₂ in the atmosphere on the other side of the silicone rubber barrier.

Polystyrene membranes have been used in tissue culture studies to provide a gas-permeable, but otherwise sealed (evaporative, sterile) environment (OptiCell™, BioCrystal Ltd., Westerville, Ohio).

CELLine™ flasks (BD Biosciences, San Jose, Calif. and Integra, Switzerland), useful for tissue culture studies, utilize a multi-component membrane technology. There is an upper semi-permeable membrane for nutrient and other small molecule passage and a molded silicone membrane that allows supplied oxygen to pass through. WO 89/11529 (filed May 19, 1989) describes a bioreactor device having a selectively permeable ultra filtration membrane, that is permeable to essential nutrients and toxic waste products.

Teplyakov (2002) Intl J of Hydrogen Energy 27:1149-1155 describes bioreactors for hydrogen production having active membrane systems. An asymmetric silicon-containing polymeric membrane, PVTMS (polyvinyltrimethylsilane), was chosen for permeability, selectivity, and physical properties.

None of the above-mentioned bioreactors utilize porous materials having an open pore structure without embedded biological material. None of the above-mentioned bioreactors utilize a porous material having an open pore structure as a structural component of the bioreactors. None of the above-mentioned bioreactors utilize porous materials having an open pore structure without also functioning as a support network for an embedded biological material. None of the above-mentioned bioreactors utilizes an aerogel, xerogel, or sol-gel glass without embedded biological material. None of the above-mentioned bioreactors utilize a porous material having an open pore structure and without embedded biological material, that is also gas permeable (by methods other than molecular diffusion), photopermeable, a sterile barrier, hydrophobic, able to be made a wide variety of three-dimensional forms, and recyclable.

All references cited are incorporated herein by reference to the extent that they are not inconsistent with the disclosure herein.

### SUMMARY OF THE INVENTION

This invention provides bioreactors comprising structural and functional elements made of selectively permeable porous materials. Porous materials useful in the practice of this invention have open pore structures. The bioreactors provided by this invention do not utilize a porous material as a support network for a cell, organism, or cellular component to be cultured. Porous materials useful in the practice of this invention are optionally selectively permeable to gases, photoradiation, visible radiation, ultraviolet radiation, cells, organisms, and/or cellular components. Porous materials useful in the practice of this invention are optionally gas permeable, photopermeable, transparent, and/or capable of functioning as a sterile barrier. Porous materials useful in the practice of this invention include, but are not limited to, aerogels, xerogels, and sol-gel glasses. Aerogels useful in the practice of this invention include hydrophobic aerogels and silica aerogels.

This invention provides bioreactors comprising porous materials as one or more portions of or entire structural or functional components of the bioreactors, including, but not limited to, walls, covers, floors, filters, windows, and tubes. Bioreactors provided by this invention include porous materials that permit fluid communication between the contents (gaseous or liquid) of the bioreactor and the atmosphere outside the bioreactor (the earth's atmosphere or a controlled atmosphere). Bioreactors provided by this invention comprise porous material forms including panels, monoliths, cylindrical vessels, cylindrical tubes, hemispheres, and portions or combinations thereof. This invention provides bioreactors comprising more than one porous material. This invention provides bioreactors having transparent and photopermeable porous materials functioning as windows.

Gas permeable porous materials useful in the practice of this invention are optionally also selectively gas permeable, and simultaneously photopermeable and/or transparent. Gas permeable porous materials are optionally permeable to acetylene, air, ammonia, argon, bromine, carbon dioxide (CO₂), carbon monoxide (CO), chlorine, ethane, ethylene, ethylene oxide, formaldehyde, helium, hydrogen, hydrogen chloride, hydrogen cyanide (HCN), hydrogen iodide, hydrogen sulfide, methane, methyl chloride, nitric oxide (NO), nitrogen, nitrous oxide (N₂O), oxygen, sulfur dioxide, gaseous fluorocarbons, sulfur dioxide, and/or volatile organic molecules. Gas permeable porous materials useful in the practice of this invention may be selectively permeable to a subset of the above gases.

This invention provides bioreactors optionally also having device elements, structural elements and/or functional elements, alone or in combination, providing a means for achieving one or more of the following processes: bioreactor sanitization and/or sterilization; monoseptic holding or processing; preventing contamination; culture agitation or circulation; temperature detection and/or control; gas delivery and/or removal; dissolved gas detection and/or control; pH detection and/or control; photoradiation delivery; detecting and/or quantitating photoradiation reception; reorienting a portion of the bioreactor relative to a source of photoradiation; liquid delivery and/or removal; nutrient detection and/or delivery; waste removal; cell and organism delivery and/or removal; gas harvesting; and harvesting product.

### BRIEF DESCRIPTION OF THE DRAWINGS

FIGS. 1A-1C illustrate a Class I bioreactor with a porous material dome cover.

FIGS. 2A-2D illustrate a Class I bioreactor with a hemispherically and cylindrically shaped porous material at the bottom, sealed with adhesives in the inlet gas piping, functioning as a filter for sparged gases, and with a second porous material sealed in the exhaust gas vent for filtering exhaust gases and preventing contamination of the bioreactor.

FIGS. 3A-3B illustrate a Class II bioreactor with a porous material cover with an optional cowl.

FIGS. 4A-4B illustrate a disposable Class II bioreactor configuration with a porous material cover.

FIGS. 5A-5C illustrate a Class III bioreactor with a porous material cover for receiving photoradiation.

FIGS. 6A-6B illustrate a Class IV bioreactor with porous material cover, with an optional photopermeable cowl section for receiving photoradiation.

FIGS. 7A-7B illustrate a Class IV bioreactor with sealed porous material cover and a porous material filter in a vent pipe, receiving photoradiation. The orientation of the bioreactor culture chamber is determined by a pivot stand.

FIGS. 8A-8B illustrate a Class IV cylindrical tube bioreactor receiving photoradiation directly and reflecting off of a parabolic photocollector which can be oriented to maximize the radiation incident on the cylindrical tube.

FIGS. 9A-9C illustrate a Class V open pond bioreactor having a tented porous material cover.

### DETAILED DESCRIPTION OF THE INVENTION

As used herein, “porous material” refers to a material having an open pore structure that is permeable to one or more gases and, in addition, is permeable to one or more of the following: photoradiation, visible radiation, ultraviolet radiation, cells, organisms, cellular components, and/or unwanted contaminants. Porous materials of this invention are not employed as a support network for a cell, organism, or cellular component to be cultured in a bioreactor of this invention. A porous material of this invention exhibits one or more of the following properties: gas permeability, selectively gas permeability, multi-directionally gas permeability. Additionally, the porous material can be photopermeable, transparent, hydrophobic, infinitely shapeable, recyclable, capable of functioning as a sterile barrier, a xerogel, an aerogel, and/or made by a sol-gel process. Porous materials useful in the practice of this invention can have any combination or sub-combination, optionally simultaneously, of the above-listed characteristics. All porous materials of this invention are useful as structural components of a bioreactor including a wall or cover, or portion thereof, or useful as functional components of a bioreactor including a window or filter, or portion thereof. Porous materials useful in the practice of this invention are not ordered atomic arrays, not crystalline (e.g., zeolites), not ceramic (e.g., ceramic membranes), and not an aggregate of otherwise non-porous particles. Porous materials useful in the practice of this invention are gas permeable through an open pore structure. Porous materials useful in the practice of this invention are gas permeable by mechanisms other than molecular diffusion.

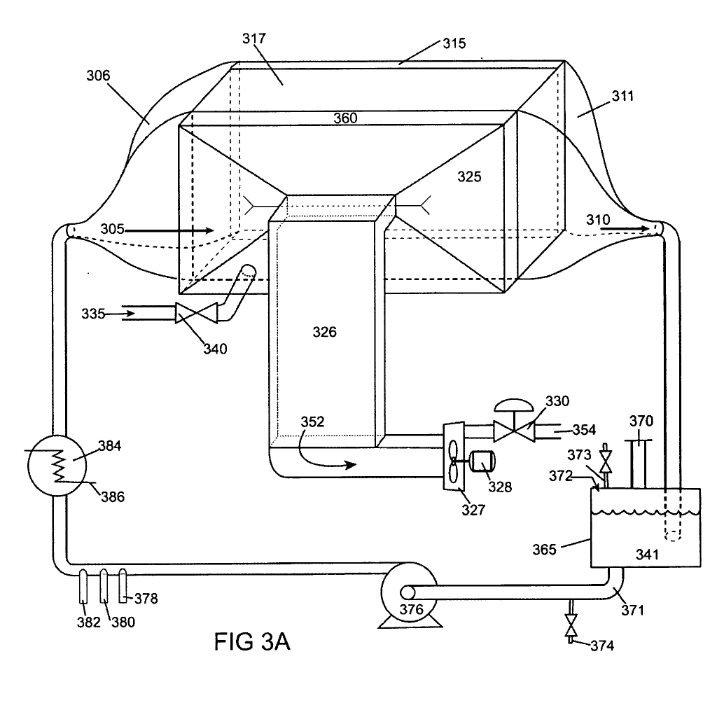

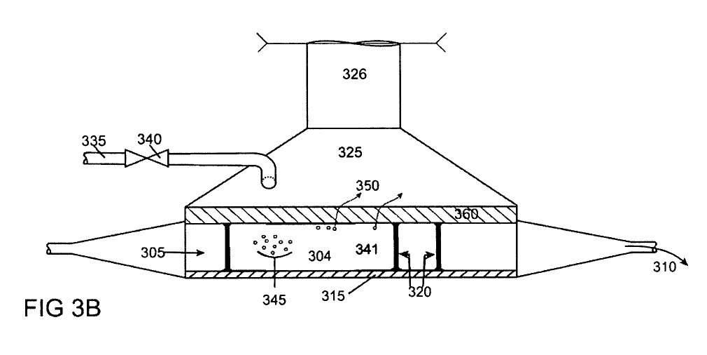

As used herein, “open pore structure” refers to an irregular interconnectedness of pores whereby gaseous fluids can flow through a material from pore to pore, and eventually substantially completely through the material, entering one side and exiting the other, wherein the pore walls are themselves porous. Photographs are useful for determining the quality and quantity of an open pore structure. A scanning electron microscope photomicrograph of an open pore structure can be found in FIG. 3 of Blaga, A. Institute for Research in Construction, Canadian Building Digest, CBD 166, 1974. “Open pore structure” is also known in the art as an open cell structure. An open pore structure is a pore structure similar to the pore structure of an aerogel. Open pore structures are not closed pore structures wherein the pore walls are not porous, are not regular crystalline structures, and are not made of regularly spaced particles that have been compacted or sintered, such as ceramics. Crystals and crystalline materials do not have open pore structures. Ceramic materials do not have open pore structures.

Methods known in the art for measuring open pore structures, porosity, and/or distinguishing open pore structures from closed pore structures, regular crystalline structures, and/or compacted or sintered structures include: Gas/Vapor adsorption (IUPAC guidelines for “Reporting Physisorption Data for Gas/Solid Systems” in Pure and Applied Chemistry, volume 57, page 603, (1985)); X-ray and neutron scattering; gas/solid NMR; electron microscopy of replicants; atomic force microscopy; light scattering (Bulk and surface light scattering from transparent silica aerogel, W. J. Platzer and M. Bergkvist, Solar Energy Materials and Solar Cells 31 (1993) 243-251; Scattering of visible light from silica aerogels, A. Beck, R. Caps and J. Fricke, J. Phys D: Appl. Phys. 22 (1989) 730-734; Optical investigations of silica aerogels, P. Wang, W. Korner, A. Emmerling, A. Beck, J. Kuhn and J. Fricke, J. of Non-crystalline Solids 145 (1992) 141-145; Light scattering for structural investigation of silica aerogels and alcogels, A. Beck, O. Gelsen, P. Wang and J. Fricke, Proceedings of the 2nd international symposium on aerogels (ISA2), Revue de physique appliquee, colloque C4, supplement au Journal de Physique 4, vol 24 (1989) 203; Scattering from microrough surfaces: comparison of theory and experiment, Y. Wang and W. L. Wolfe, J. Opt. Soc. Am. vol. 73, no. 11 (1983) 1596); positron lifetime annihilation spectroscopy (J. N. Sun, Y. F. Hu, W. E. Frieze, and D. W. Gidley, Characterizing Porosity in Nanoporous Thin Films Using Positronium Lifetime Annihilation Spectroscopy, Radiation Physics and Chemistry, 68, 345 (2003); T. L. Dull, W. E. Frieze, D. W. Gidley, J. N. Sun and A. F. Yee, Determination of Pore Size in Mesoporous Thin Films from the Annihilation Lifetime of Positronium, The Journal of Physical Chemistry B 105, 20, 4657 (2001); and D. W. Gidley, T. L. Dull, W. E. Frieze, J. N. Sun and A. F. Yee, Probing Pore Characteristics in Low-K Thin Films Using Positronium Annihilation Lifetime Spectroscopy, Materials Research Society Symposium Proceeding 612, D4.3.1 (2000)); mercury porosimetry and helium pycnometry (Pore Structure of Different LWAs, Faust and Beck, Lacer No. 4 1999, pp 123-132).

As used herein, “porous material having an open pore structure” and “open pore structured porous material” refer to aerogels and other materials having pore structures that are similar to the pore structures of aerogels, including, but not limited to xerogels and sol-gel glasses. The pores are interconnected and irregular, i.e. not of a regular repeating pattern as is found in a crystal or a crystalline material.

As used herein, “pore” refers to an opening in a material. At the position when the diameter substantially changes, a new pore begins. The International Union of Pure and Applied Chemistry has recommended a classification for porous materials where pores of less than 2 nm in diameter are termed “micropores”, those with diameters between 2 and 50 nm are termed “mesopores”, and those greater than 50 nm in diameter are termed “macropores”. Aerogels, including silica aerogels, often possess pores of all three sizes. However, the majority of the pores fall in the mesopore regime, with relatively few micropores.

Porous material pores are preferably nanoscale in cross-sectional diameter, typically with a pore size of less than about 1000 nm, less than about 500 nm, less than about 200 nm, more than about 0.1 nm, or more than about 1 nm. Porous materials can have pores between about 1 nm to about 500 nm, between about 2 nm to about 250 nm, between about 3 nm to about 100 nm, or between about 5 nm to about 50 nm.

As used herein, “sol-gel process” refers to a process for forming a material at a low temperature by chemical polymerization of precursors in a liquid phase to form a gel. As used herein, “sol-gel glass” is a glass made by a sol-gel process. As used herein, “xerogel” refers to a dried out material that has passed through a gel stage or a sol-gel glass stage during preparation.

As used herein, the term “aerogel” is used as broadly as it is in the art and includes a material having a continuous solid phase containing dispersed gas in an open pore structure. Aerogels typically are xerogels, that is they pass through a gel stage during preparation. Aerogel pores are nanoscale in cross-sectional diameter, typically less than 999 nm, more than 0.1 nm, between about 1 nm to about 500 nm, between about 2 nm to about 250 nm, between about 3 nm to about 100 nm, or between about 5 nm to about 50 nm. Aerogels have porosities in the range from about 50% to about 99.9%, typically above 90%. Aerogels comprising one or more polymers are known in the art. Composite aerogels are known in the art. Modifiers can be added during preparation to modify the resulting properties of the aerogel. Organic materials that do not become embedded proteins, cells, or organisms that are intended to be cultured to make the selected product may be added to porous materials useful in the practice of this invention.

As used herein, “silica aerogel” refers to an aerogel that is produced using a silica-containing precursor. As used herein, a “photopermeable silica aerogel” refers to a silica aerogel produced with enough photopermeability-contributing silica-containing precursor monomers and few enough photopermeability-interfering ingredients wherein the resulting aerogel is photopermeable.

As used herein, “zeolite” is used as in the art and includes a crystalline, porous aluminosilicate, having regular, crystalline, but not open pore channels. Zeolite pores are of about 2-12 angstroms in diameter. Zeolites do not have an open pore structure.

As used herein, “selectively permeable” refers to the ability of selected molecules, particles, waves, compounds, compositions, gases, photoradiation, visible radiation, ultraviolet radiation, hydrophobic liquids, hydrophilic liquids, cells, organisms, cellular components, and/or unwanted contaminants to permeate or migrate through a given material, but not through another. For example, one or more gases (permeable gases) may permeate through a selectively permeable material, while one or more different gases (non-permeable gases) do not. More broadly, the selectively permeable material may exhibit higher permeability of a first gas than a second gas. In another example, one or more gases may permeate through a selectively permeable material, while cells and microorganisms do not.

As used herein, “gas permeable” refers to the ability of a material to allow gases to permeate through the open pore structure of a material. Gas permeable materials may or may not be selectively gas permeable. Gases are able to permeate through materials by methods including, but not limited to, hydraulic flow (both laminar and turbulent), convection (forced and natural), Knudsen diffusion, and molecular diffusion (also known as Fickian diffusion). Porous materials useful in the practice of this invention are gas permeable as a result of the open pore structure, and by a method other than molecular diffusion. As used herein, “selectively gas permeable” refers to the ability of a material to be more permeable to one material, compound, or molecule, relative to another material, compound, or molecule.

The term “fluid communication” is used herein to indicate the passage of one or more gases or vapors, and the term is used to indicate that a porous material of this invention provides for passage of one or more gases or vapors. Typically fluid communication is provided between the inside of a bioreactor and the environment outside of the bioreactor which may be an environment that is controlled (e.g., a gas source). Fluid communication may be selective, i.e., the material providing fluid communication may be selectively permeable. Porous materials preferred for use in this invention are those that provide for fluid communication without substantial passage of liquids, such as aqueous medium. Porous materials preferred for use in this invention are those that permit passage of one or more gases or vapors between the contents (gaseous or liquid) of the bioreactor and the atmosphere outside the bioreactor (the earth's atmosphere or a controlled atmosphere)

As used herein, “photopermeable” refers to being adequately permeable to photoradiation of the proper wavelength(s) to provide for maintenance and/or growth of a culture, particularly of cultures of photosynthetic organisms and/or cells, or for utilization of a selected biochemical reaction or pathway. As used herein, “photoradiation” refers to electromagnetic radiation of sufficient quantity and of the proper wavelength(s) to provide for maintenance and/or growth of a culture, particularly of cultures of photosynthetic organisms and/or cells, or for utilization of a selected biochemical pathway. As used herein, “transparent” refers to a material being adequately permeable to visible radiation to allow a human eye to see through it and/or to being adequately permeable to ultraviolet radiation or other light wavelengths capable of sterilizing a bioreactor of this invention.

As used herein, “hydrophobic” refers to water and other hydrophilic liquids not being able to enter pores in a porous material. A “hydrophobic porous material” will not take up water into its pores. A porous material can be hydrophobic as a result of the presence of hydrophobic moieties at its surface(s). The hydrophobicity of a material with pores can be overcome by increasing the pressure on a liquid to overcome the capillary repulsive forces that would normally prevent water from being taken up into the pores. It is preferred that methods that overcome the hydrophobicity of aerogels in bioreactors of this invention are not used in the practice of this invention. A “hydrophobic porous material” refers to a porous material that does not take up water during the environmental conditions that exist when utilizing a bioreactor of this invention.

As used herein, “bioreactor” refers to a reactor vessel, including an open vessel, for culturing one or more cells or organisms, or for maintaining cellular components, including proteins and organelles. As used herein, “vessel” refers to a device useful for containing a liquid by a method other than by embedding or encapsulating it. As used herein, a vessel includes any device useful for containing by a method other than embedding, including an open pond bioreactor (Class V) dug out of the earth. As used herein, “photobioreactor” refers to a bioreactor capable of culturing or maintaining a culture that requires photoradiation to produce a selected product. A photobioreactor has a means for receiving and delivering photoradiation to the culture, optionally from an artificial photoradiation source within (and part of) or outside of the photobioreactor or from a natural source such as the sun. A photopermeable material can be utilized between the photoradiation source and the culture. Bioreactors can perform (or include means to perform) many other functions, including, but not limited to: sanitization and/or sterilization (creating a clean or sterile space or volume within the vessel apart from the outside environment); preventing contamination; culture agitation or circulation; temperature detection and/or control; gas delivery and/or removal; dissolved gas detection and/or control; pH detection and/or control; detecting and/or quantitating photoradiation reception; reorienting a portion of the bioreactor relative to a source of photoradiation; liquid delivery and/or removal; nutrient detection and/or delivery; waste removal; cell, organism, or cellular component delivery and/or removal; gas harvesting; and/or harvesting product. Different classes of bioreactors are discussed above.

As used herein, “wall” refers to a layer of material functioning as a structural component of a reactor, including side walls, top walls, bottom walls, interior and exterior walls, and walls having more than one of the above-listed orientations or locations. Walls can be vertical or horizontal, or have components of each. A wall has two primary surfaces and one or more edges. As used herein, “cover” refers to a top wall or a lid, that has a horizontal component and that is capable of covering. A cover is usually selectively removable. A wall can also function as a bottom or a floor. As used herein, “cowl” is a cover often utilized as a hood for directing something. In bioreactors of this invention, cowls can be utilized to direct gases.

As used herein, “panel” refers to a form having three dimensions, one of which is significantly smaller relative to the other two dimensions. A panel can be flat or not flat. A panel can be made up of many smaller panels. A panel may be oriented horizontally, vertically, or with horizontal and vertical components. A panel may be utilized as a wall. A thick panel, i.e., the small dimension is closer in magnitude to the other two dimensions, is called a monolith. A panel can be photopermeable. A photopermeable panel of a bioreactor can be functionally connected to a means for orienting it relative to a source of photoradiation. As used herein, “cylindrical” refers having the form of a generalized cylinder (Gray, A. Modern Differential Geometry of Curves and Surfaces with Mathmatica, 2^{nd} ed. Boca Raton, Fla., CRC Press, pp 439-441; Harris J. W. and Stocker H, “General Cylinder” Section 4.6.1 in Handbook of Mathematics and Computational Science, NY, Springer Verlag, p 103, 1998; and Kern W. F. and Bland J. R. “Cylindrical Surface” Section 14 in Solid Mensuration with Proofs, 2^{nd} ed., NY, Wiley, pp 32-36, 1948).

As used herein, “filter” refers to a material that is selectively permeable.

Walls, panels, covers, lids, vessels, chambers, filter, tubing, conduits and the like are examples of structural elements of a bioreactor. Structural elements may have additional functions. Herein, function or additional function can be imparted to a structural element by making or forming the structural element or a portion thereof from a porous material of this invention or providing or attaching a porous material of this invention to a structural element.

As used herein, “providing remaining bioreactor components” refers to providing, in addition to a provided porous material, all other components necessary for assembling a selected bioreactor, as is known in the art.

As used herein, “cell” includes all cells and cell-like units of life including, but not limited to, prokaryotes, eukaryotes, viruses, bacteria, animals cells, plant cells, fungal cells, algal cells, and engineered cells. A material of this invention may be selectively permeable to certain cells and cell-like units and not to others.

As used herein, “maintaining cellular components” refers to providing environmental conditions whereby cellular components are able to perform one or more selected functions, including performing one or more steps of a biochemical pathway, resulting in the production of a product. As used herein, “culturing” or “to culture” refers to providing environmental conditions whereby one or more cells, organisms, or cellular components are able to perform one or more selected functions, e.g., performing one or more steps of a biochemical pathway, growth, or reproduction, whereby the direct or indirect goal is production of a product. Products include gases, biomass, chemicals, and pharmaceuticals. As used herein, “culture” refers to a cell, organism, or cellular component, and optionally a culture medium in which the cells, organisms or cell components are cultured, maintained, or grown. As used herein, “culture medium” and “culture fluid” refer to the liquid and optionally nutrients within the liquid in which a cell, organism, or cellular component exists for maintenance, culture, or growth. As used herein, “environmental conditions” refers to all elements of a cell, organism, or cellular component environment in a bioreactor including but not limited to temperature, gas pressure and composition, nutrient and waste quantity and quality (including as solid, liquid, and/or gas), pH, and photoradiation quantity and quality.

As used herein, “sterile barrier” refers to a material barrier capable of preventing substantial transfer of cells and organisms and thereby preventing undesired contamination. A sterile barrier is capable of keeping desired cells, organisms, cell-like units and/or cellular components on one side and undesired cells, organisms, and/or cellular components on another side. A sterile barrier is a type of filter. Porous materials act as sterile barriers by size exclusion. In a material having an open pore structure, the material can act as a sterile barrier because the pore size is sufficiently small. As used herein, “substantial transfer” is transfer that interferes with the growth, maintenance, and/or biochemical function of a culture, or production of a selected product by the culture. Some sterile barriers can prevent transfer of all cells and organisms. If a sterile barrier has an open pore structure, the pore size is small enough to exclude the smallest relevant cells, organisms, and cellular components that are selected to remain in or out of the bioreactor. Sterile barriers that function by size exclusion have a pore size of equal to or less than about 200 nm. As used herein, “pore size” refers to the largest size composition that can permeate a material having an open pore structure. A porous material having a selected pore size is selectively permeable to objects up to the pore size. An open pore structure material having a pore size of about 200 nm can have pores that have a diameter larger than about 200 nm, however the pores that are larger than 200 nm are sufficiently widely distributed and of a sufficiently short length that they do not cross through an entire dimension of the material alone nor in connection with other pores larger than about 200 nm, whereby the connected pores are uninterrupted by pores having a diameter smaller than about 200 nm. Open pore structured porous materials that have a pore size of 200 nm are impermeable to compositions larger than 200 nm. As used herein, “monoseptic” and “monoseptic conditions” refer to when only one or more selected cells, organisms, or cellular components are present, and other non-selected cells, organisms, and cellular components are prevented from entering into the monoseptic environment. A bioreactor of this invention that provides a monoseptic environment does not allow any contaminating cells, organisms, or cellular components into the bioreactor.

As used herein, “embedded” refers to a composition being incorporated or encapsulated in a material such that the composition becomes part of a new combined material, in contrast to a composition being surrounded by material, as contents are surrounded by their container or vessel. The material in which a composition is embedded functions as a support network of the embedded composition. As used herein, “support network” refers to a structural support having a protein, cell, or organism embedded within. Vessels and containers provide support, but they are not support networks.

In specific embodiments, this invention provides bioreactors having porous materials having a pore size of about 500 nm or about 200 nm. This invention provides bioreactors having porous materials having pores having diameters greater than about 0.5 nm. This invention provides bioreactors having porous materials having pore diameters between about 1 nm and about 500 nm, between about 2 nm and about 250 nm, between about 3 nm and about 100 nm, or between about 5 nm and about 50 nm.

In specific embodiments, this invention provides bioreactors comprising porous materials made by a sol-gel process.

In specific embodiments, this invention provides a method for making a bioreactor of this invention comprising: providing a porous material; providing remaining bioreactor components; and assembling the porous material and the components. The porous material can be selected to have a pore size appropriate for the intended application.

In specific embodiments, this invention provides a method for culturing a cell comprising: providing a bioreactor of this invention; providing a cell; and providing environmental conditions whereby the cell is cultured. The bioreactors of this invention are capable of culturing cells that are viral, bacterial, plant, fungal, algal, or animal cells, including mammalian cells. This invention provides a method for culturing a cell of Chlamydomonas reinhardtii, Chlamydomonas moewusii, Chlamydomonas sp. strain MGA161, Chlamydomonas eugametos, and Chlamydomonas segnis belonging to Chlamydomonas; Chlorella vulgaris belonging to Chlorella; Senedesmus obliguus belonging to Senedesmus; and Dunaliella tertrolecta belonging to Dunaliella, Anabaena variabilis ATCC 29413 belonging to Anabaena, Cyanothece sp. ATCC 51142 belonging to Cyanothece, Synechococcus sp. PCC 7942 belonging to Synechococcus, Anacystis nidulans belonging to Anacystis, Rhodopseudomonas palustris and Rhodopseudomonas acidophila belonging to Rhodopseudomonas, and Rhodospirillum rubrum ATCC 11170, Rhodospirillum rubrum IFO 3986 belonging to Rhodospirillum, Rhodobacter sphaeroides, Rhodobacter capsulatus ATCC 23782, ATCC 17013 belonging to Rhodobacter, and Rhodovulum strictum, Rhodovulum adriaticum, Rhodovulum sulfidophilum belonging to Rhodovulum, purple nonsulfur bacteria belonging to Rhodospirillaceae, or green gliding bacteria belonging to Chloflexaceae.

In specific embodiments, this invention provides a method for culturing a cell that produces a product, wherein products are not limited to, but include hydrogen gas, biomass, chemicals, and pharmaceuticals.

In specific embodiments, this invention provides a method for culturing an organism comprising: providing a bioreactor of this invention; providing an organism; providing nutrients for the organism; adding the nutrients and the organism to the bioreactor; and providing environmental conditions whereby the organism is cultured. The method for culturing organisms is useful for culturing plants.

In specific embodiments, this invention provides a method for producing hydrogen gas comprising: providing a bioreactor of this invention; providing a hydrogen-producing cell or organism; and providing environmental conditions whereby the cell or organism produces hydrogen.

In specific embodiments, this invention provides a method for producing a product selected from the group consisting of gaseous products, biomass, chemicals, and pharmaceuticals, the method comprising: providing a bioreactor of claim 1; providing a cell, organism, or cellular component capable of producing the product; providing environmental conditions whereby the cell, organism, or cellular component produces the product; and allowing the cell or organism to produce the product.

Additional embodiments of this invention are exemplified in the figures.

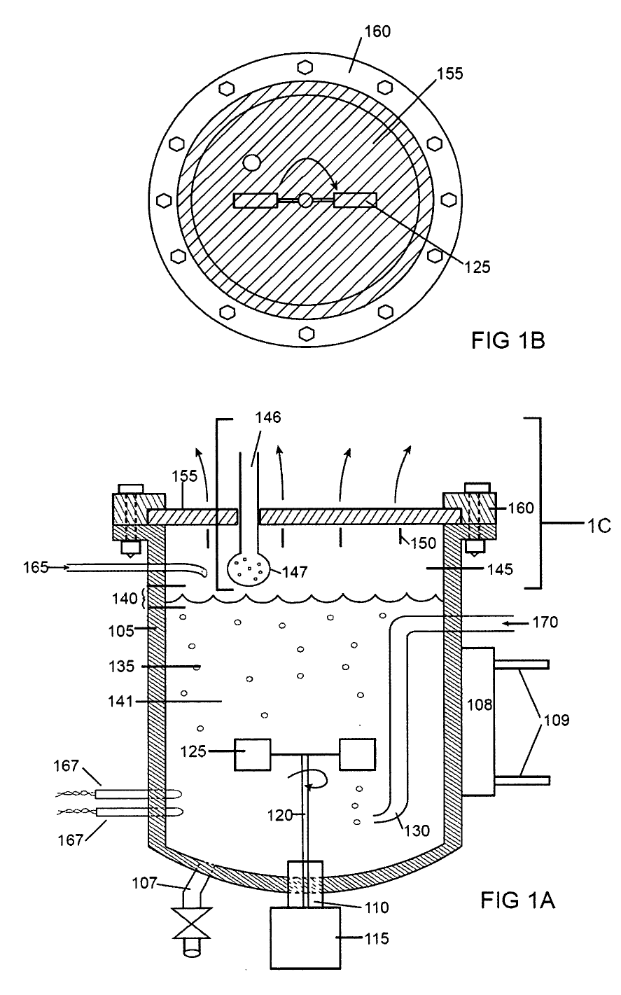

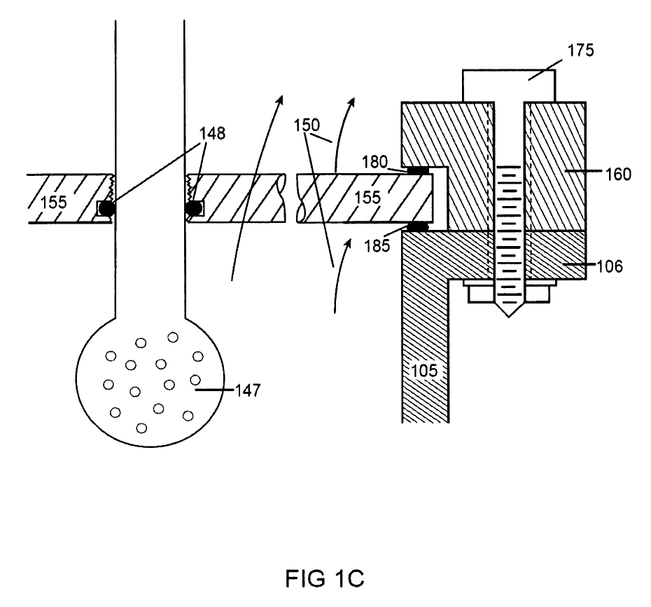

FIGS. 1A-1C illustrate a Class I bioreactor comprising a porous material cover (top wall) lid 155. FIG. 1A illustrates a cross-sectional lateral view of the reactor. FIG. 1B illustrates a top view of the reactor. FIG. 1C illustrates a close up of the gasket and O-ring seal details from FIG. 1A, as shown in brackets. The bioreactor illustrated in FIGS. 1A-1C has a vessel wall 105 that extends from a curved bottom to a wide top rim 106. The vessel wall 105 has various inlet and outlet ports for a vessel drain and drain valve 107, agitator components (agitator drive motor 115, agitator shaft 120, a double mechanical seal 110, and a gas dispersion impeller 125), a sparge gas inlet port 170 with sparge tube 130, an addition port tube 165, and optional environmental probes 167. The vessel wall 105 is heat transferably connected to a vessel jacket for heating and cooling 108 which has a means for receiving in and delivering out heating/cooling media 109. The vessel wall 105 contains medium 141 up to a liquid level 140, which is below a gas headspace 145. The wide top rim 106 is sealed to a porous material monolith 155 which functions as the vessel cover (top wall) 155. The cover (top wall) 155 is held in place by a cover (top wall) sealing ring 160 and a retaining fastener 175, and is sealed by an upper sealing gasket 180 and a lower sealing gasket 185. The porous material cover (top wall) 155 has a port for a spray distribution ball 147 for cleaning and sterilizing solutions which is sealed to the porous material cover (top wall) 155 with an O-ring seal 148 between the spray ball feed pipe 146 and porous material monolith 155. Gas bubbles 135 are shown dispersed within the liquid medium 141. When the bioreactor illustrated in FIGS. 1A-1C is in use, sparged gases bubble up 135 through the media 141 to the gas headspace 145. Gases in the headspace 145 exit 150 the bioreactor through the porous material monolith cover (top wall) 155. The porous material cover (top wall) lid 155 is made of a porous material that is gas permeable to all gases that must be vented from the bioreactor and that is of an open pore structure capable of functioning as a sterile barrier. All connections and seals are capable of maintaining monoseptic conditions, as necessary.

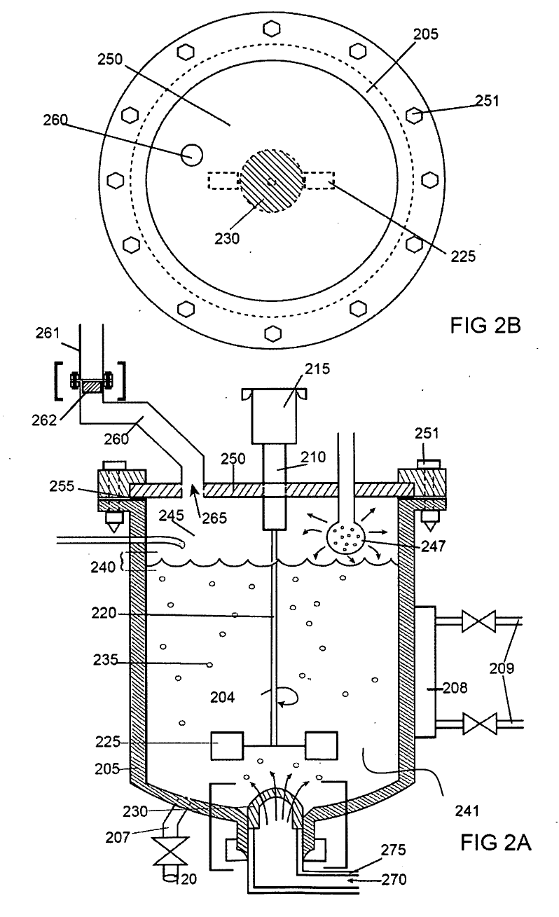

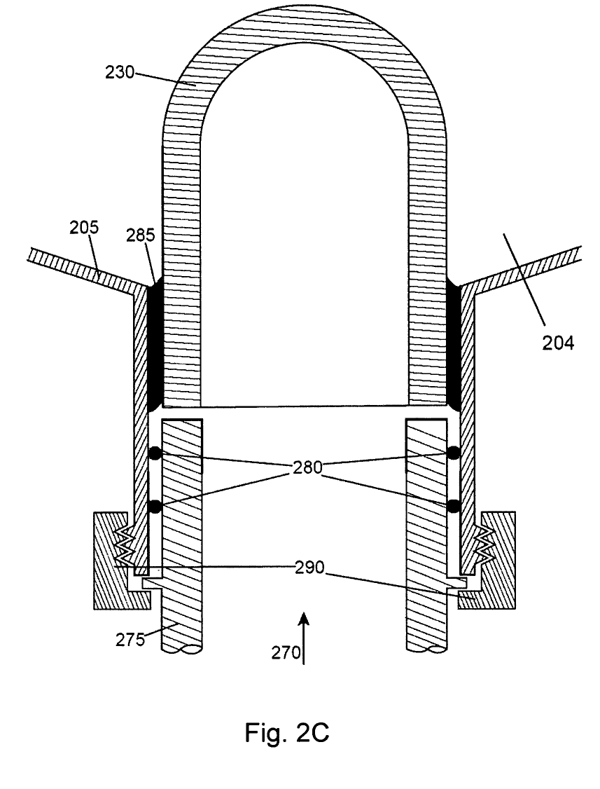

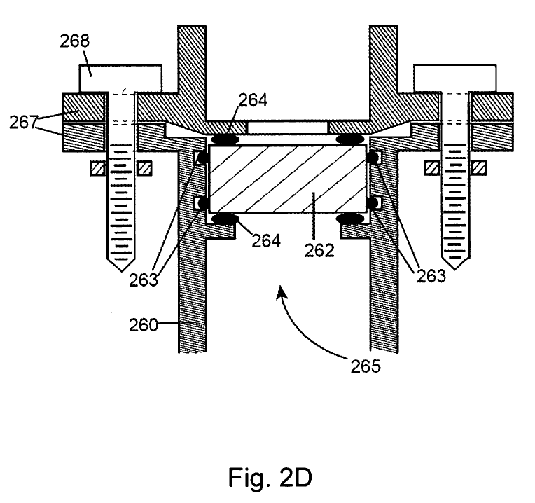

FIGS. 2A-2D illustrate a Class I bioreactor comprising a porous material dome 230 at the gas entry point and a porous material filter 262 at the gas exit point. FIG. 2A illustrates a cross-sectional side view of the bioreactor. FIG. 2B illustrates a top view of the bioreactor with the diagonal line shading showing the location of the porous material dome 230 within the reactor. FIG. 2C illustrates a detailed cross-sectional side view of the porous material dome 230 mounted into the bioreactor, shown in brackets in FIG. 2A. FIG. 2D illustrates a detailed cross-sectional side detailed view of the exhaust gas vent 260, also designated by brackets in FIG. 2A. The bioreactor has a vessel wall 205 that extends from an angled bottom to a rimmed top. The top of the vessel wall 205 is closed by a vessel cover 250. The vessel cover 250 and the vessel wall 205 are sealed with a sealing gasket 255 and one or more fasteners 251. The vessel wall 205 has various inlet and outlet ports for an incoming gas duct 275, vessel drain and drain valve 207, and for additions 265. The vessel cover 250 is pierced with ports for agitator components (agitator drive motor 215, double mechanical seal 210, agitator shaft 220, and gas dispersion impeller 225), a spray distribution ball 247 for cleaning and sterilizing solutions, and an exhaust vent 260. The vessel wall 205 is heat transferably connected to a vessel jacket for heating and cooling 208 which has a means for receiving in and delivering out heating/cooling media 209. The vessel wall 205 contains medium 241 up to a liquid level 240, which is underneath a gas headspace 245. The incoming gas duct 275 is sealed to the interior side of the vessel wall 205 by a locking ring 290 and O-ring seals 280. A cylindrical/hemispherical porous material dome 230 through which sparge gases 270 pass upon entry into the interior of the bioreactor culture chamber 204 is sealed by an adhesive seal 285 to the interior side of the vessel wall 205 outside of the culture chamber, more internally relative to the incoming gas duct 275 and protruding into the culture chamber 204. Gas bubbles 235 are shown dispersed within the culture medium 241. The exhaust gas vent 260 extends away from the culture chamber 204. The inside wall of the exhaust gas vent 260 is sealed with O-rings 263 to a second porous material 262 that acts as a filter for exit gases thereby preventing contamination of the culture chamber. The exhaust gas vent has a flange 267 beyond the second porous material 262 and seal 263, that is operationally connected to an upper exhaust duct 261 with a fastener 268. The doughnut shaped gasket 264 allows the upper part of flange 267 to push against the porous material filter 230 without breaking it (as might occur if metal pushed directly against the porous material). A second gasket 264 is beneath the second porous material 262. When the bioreactor illustrated in FIGS. 2A-2D is in use, sparge gases enter 270 through the incoming gas duct 270, pass through the porous material 230 into the culture medium 241, and form bubbles 235. The bubbles rise to the surface of the liquid level 240, join the gas headspace 245, and gases are exhausted through 265 the exhaust gas vent, where they pass through the porous material 262. The porous material dome 230 is made of a porous material that is gas permeable to all gases that must enter the bioreactor and that is of an open pore structure capable of functioning as a sterile barrier. The second porous material filter 262 is made of a porous material that is gas permeable to all gases that must exit the bioreactor and that is of an open pore structure capable of functioning as a sterile barrier. All connections and seals are capable of maintaining monoseptic conditions, as necessary.

FIGS. 3A and 3B illustrate an angled overhead perspective and a front cross-sectional view, respectively, of a Class II bioreactor with a porous material cover 360. FIG. 3B provides more detail of a section of the bioreactor of FIG. 3A. The bioreactor has bottom backplate 315 operationally connected to one or more retaining walls 317. The retaining walls 317 are operationally connected to a porous material cover 360, which is operationally connected to a negative pressure cowl 325. The edges of the retaining walls 317, bottom backplate 315, and porous material cover 360 are operationally connected on an inlet side to an inlet manifold 306 where a live culture enters 305 the culture chamber 304 and on an outlet side to an outlet manifold 311 where a live culture exits 310 the culture chamber 304. One or more support pillars 320 are operationally connected to the backplate 315 and the porous material cover 360. The negative pressure cowl 325 is operationally connected to a suction plenum for gases 326 which is operationally connected to a blower or gas fan 327 driven by a motor 328. The blower 327 is also operationally connected to a harvest gas pressure flow control valve 330. The cowl 325 is pierced by a port for a gas inlet 335 with an inlet gas flow control valve 340. The outlet manifold 311 is operationally connected to a liquid reservoir 365 into which medium 341 is delivered. The liquid reservoir 365 has an organism/cell and nutrient inlet port 372, a liquid fill port 373, and an alternative gas vent 370. The alternative gas vent 370 provides another way for gases to leave the bioreactor, for example when adding liquid volume. For gas bubbles that are generated but that stochastically don't make it to the aerogel cover (top wall) 360, the vent 370 provides another means for harvesting product gases. Vent 370 can optionally be connected to the negative pressure plenum 326 leading to the blower or gas fan 327. The liquid reservoir in the vent 370 provides a space whereby liquid inventory can rise and fall, if necessary, depending on culture conditions. The presence of this liquid reservoir ensures that the section of the bioreactor containing the porous material cover 360 can always be 100% liquid full. The reservoir is located at the hydraulic high point of the system, and the pump 376 is at the hydraulic low point of the system. The liquid reservoir is operationally connected to the an inlet duct 371 which is operationally connected to a bioreactor low point drain and drain valve 374, a recirculation pump 376, a pH probe 378, a dissolved gas probe 380, a temperature probe 382, and a heat exchanger 384 for controlling culture medium 341 temperature using a cooling/heating medium 386. The inlet duct is operationally connected to the inlet manifold 306. The culture medium 341 is circulated through the bioreactor.

When Chlamydomonas is cultured for hydrogen production, an inert gas that does not interfere with hydrogen production is introduced into the cowl through the gas inlet 335, until the culture medium has about zero dissolved oxygen. The dissolved oxygen migrates through the porous material to reach equilibrium with the atmosphere containing the inert gas until essentially no dissolved oxygen remains in the culture medium. The culture produces hydrogen gas shown as bubbles 345 in the medium 341. The bubbles 345 rise and the gas migrates through 350 the porous material cover 360 due in part to the negative pressure within the cowl 325. The gas is pulled towards 352 the blower or gas fan 327 through the harvest gas pressure control valve 330 for collection 354. The porous material cover 360 is made of a porous material that is gas permeable to hydrogen and all other gases that must be vented from the bioreactor and that is of an open pore structure capable of functioning as a sterile barrier unless monoseptic conditions are maintained by other mechanisms. All connections and seals are capable of maintaining monoseptic conditions, as necessary.

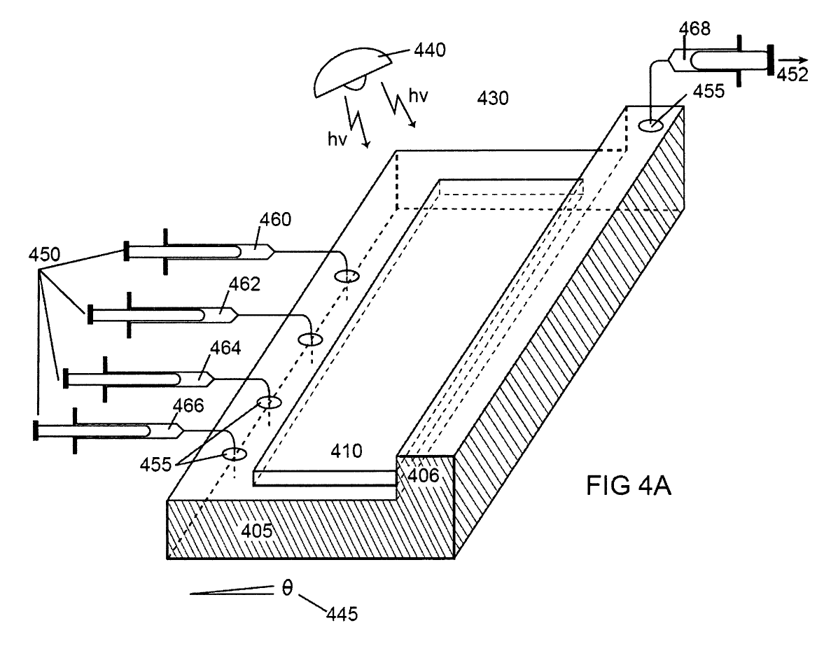

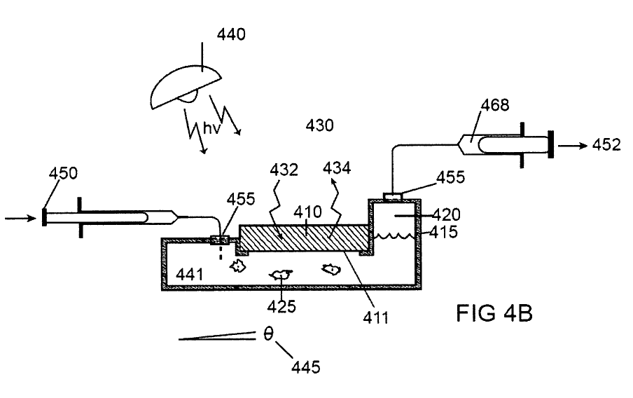

FIGS. 4A and 4B illustrate an angled overhead perspective and a cross-sectional side view respectively, of a Class II bioreactor with a porous material monolith window 410 which also serves as a cover. The bioreactor has a main body 405 that functions as a vessel for containing a medium 441 for cells or organisms 425. The main body 405 has an opening that is sterile-sealed with a porous material window 410. The medium 441 is at a liquid level 415 about equal with or higher than the porous material window 410 bottom surface 411 so that the medium 441 is in liquid contact with the entire porous material window 410. The main body also has septum ports 455 for syringes useful for adding cell suspension for inoculation 460, initial growth medium 462, special nutrients 464, and induction compound 466. The main body 405 has an elevated side 406 above the porous material window 410 that contains a gas headspace 420 above the medium 441. A septum port 455 in a top corner of the elevated side 406 accepts a syringe for withdrawing 468 gas from the gas headspace 420 to make room for liquid volume additions or for removing liquid volume from the bioreactor. The syringes for adding are pushed in 450 to add materials and the syringe for withdrawing 468 is pulled out 452 to make room for liquid volume additions. Preferably the withdrawing syringe is pulled out 452 about simultaneously with the pushing in 450 of the adding syringe(s) to prevent an increase of the pressure on the porous material window 410. The septum ports 455 are sterile-sealed for use with syringes, as is known in the art. The bioreactor main body 405 can optionally be tipped at an angle θ 445 when filling the bioreactor main body 405 with medium 441 to allow the medium to be in direct liquid contact with the entire bottom surface 411 of the porous material window 410, without gas bubbles. The gas outside of the bioreactor 430 can be ambient air or a selected gas composition in an incubator. When the selected organism or cell 425 to be cultured in the bioreactor requires illumination, the porous material window 410 is transparent and an illumination source 440 provides illumination (hν) for viewing the culture and/or sterilizing the bioreactor. Optionally the porous window 410 is photopermeable and the illumination source 440 is a photoradiation source. The porous material window 410 is two-way gas permeable allowing gases to migrate from the gas outside the bioreactor 430 through the porous material into 432 the medium 441 and gases to migrate from the medium through the porous material out 434 of the bioreactor. If the selected cells or organisms 425 undergo photorespiration (if used as a Class IV bioreactor) within the bioreactor, oxygen can migrate from the outside air 430 through the porous material into 432 the bioreactor and the medium 441 and carbon dioxide produced by the cells/organisms 425 can migrate from the medium 441 through the porous material out 434 of the bioreactor. The porous material window 410 is made of a porous material that is photopermeable, gas permeable to all gases that must be vented in and out of the bioreactor for production of the selected product, and that is of an open pore structure capable of functioning as a sterile barrier. All connections and seals are capable of maintaining monoseptic conditions, as necessary. The porous material cover optionally provides a location for anchorage for mammalian or plant cell or insect cell cultures requiring such an anchorage.

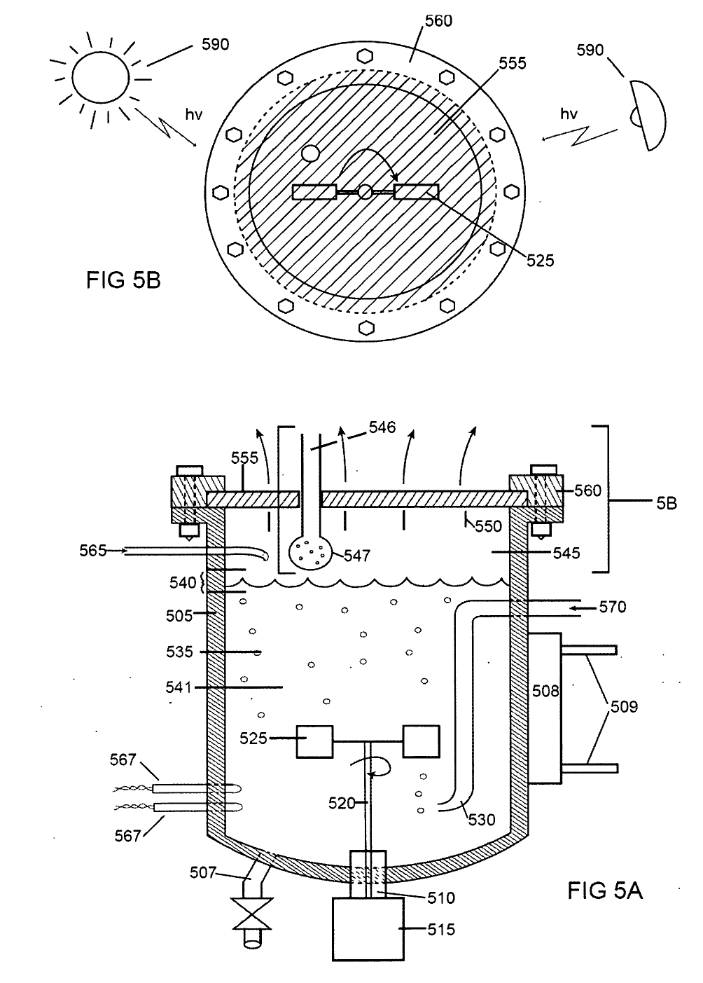

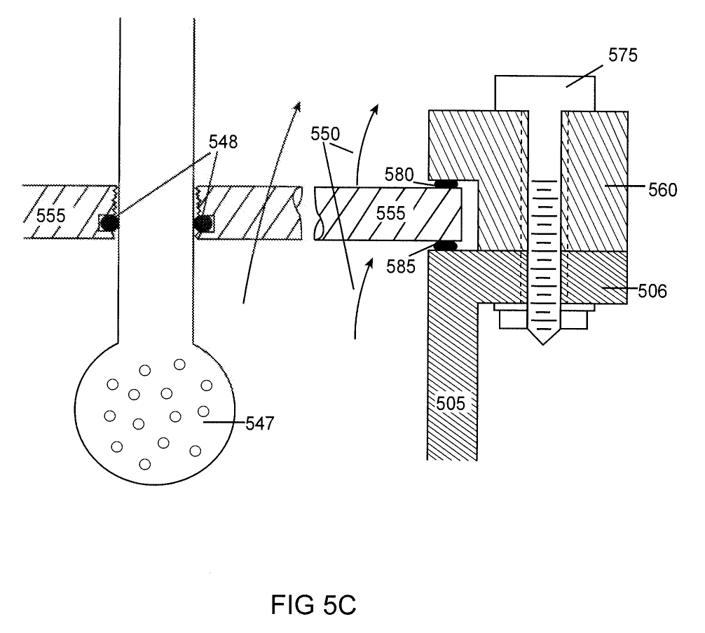

FIGS. 5A-5C illustrate a Class III bioreactor comprising a porous material cover (top wall) lid 555. FIG. 5A illustrates a cross-sectional lateral view of the reactor. FIG. 5B illustrates a top view of the reactor. FIG. 5C illustrates a close up of the gasket and O-ring seal details from FIG. 5A, as shown in brackets. The bioreactor illustrated in FIGS. 5A-5C has a vessel wall 505 that extends from a curved bottom to a wide top rim 506. The vessel wall 505 has various inlet and outlet ports for a vessel drain and drain valve 507, agitator components (agitator drive motor 515, agitator shaft 520, a double mechanical seal 510, and a gas dispersion impeller 525), a sparge gas inlet port 570 with sparge tube 530, an addition port tube 565, and optional environmental probes 567. The vessel wall 505 is heat transferably connected to a vessel jacket for heating and cooling 508 which has a means for receiving in and delivering out heating/cooling media 509. The vessel wall 505 contains medium 541 up to a liquid level 540, which is below a gas headspace 545. The wide top rim 506 is sealed to a porous material monolith 555 which functions as the vessel cover (top wall) 555. The cover (top wall) 555 is held in place by a cover (top wall) sealing ring 560 and a retaining fastener 575, and is sealed by an upper sealing gasket 580 and a lower sealing gasket 585. The porous material cover (top wall) 555 has a port for a spray distribution ball 547 for cleaning and sterilizing solutions which is sealed to the porous material cover (top wall) 555 with an O-ring seal 548 between the spray ball feed pipe 546 and porous material monolith 555. Gas bubbles 535 are shown dispersed within the liquid medium 541. Photoradiation is supplied by a natural or an artificial source 590. When the bioreactor illustrated in FIGS. 5A-5C is in use, sparged gases bubble up 535 through the media 541 to the gas headspace 545. Gases in the headspace 545 exit 550 the bioreactor through the porous material monolith cover (top wall) 555. The porous material cover (top wall) lid 555 is made of a porous material that is gas permeable to all gases that must be vented from the bioreactor and that is of an open pore structure capable of functioning as a sterile barrier. The porous material monolith cover (top wall) 555 is photopermeable. All connections and seals are capable of maintaining monoseptic conditions, as necessary.

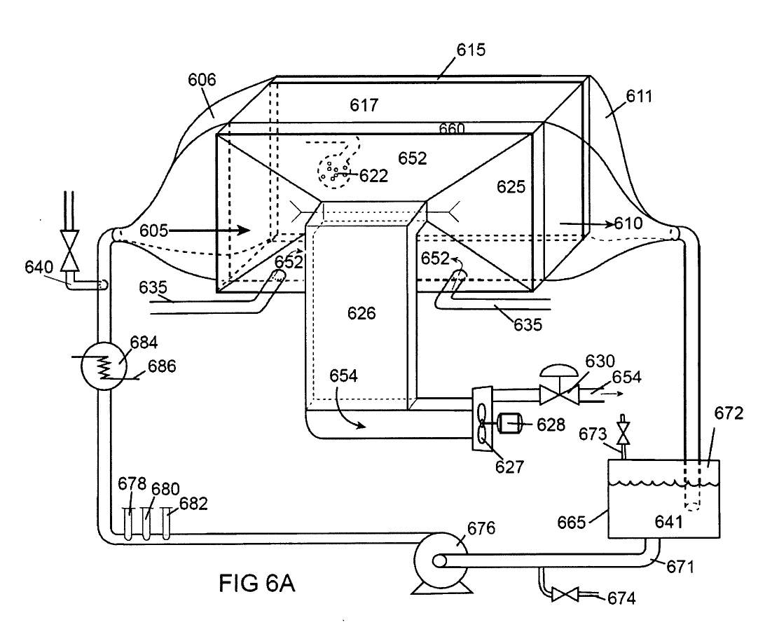

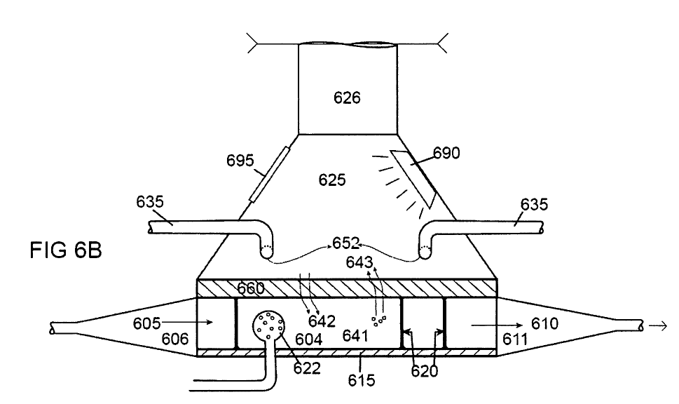

FIGS. 6A and 6B illustrate an angled overhead perspective and a front cross-sectional view, respectively, of a Class IV bioreactor with a porous material cover 660. FIG. 6B illustrates a portion of the bioreactor of FIG. 6A in more detail. The bioreactor has a bottom backplate 615 operationally connected to one or more retaining walls 617. The retaining walls 617 are operationally connected to a porous material cover 660, which is optionally operationally connected to a negative pressure cowl 625. The edges of the retaining walls 617, bottom backplate 615, and porous material cover 660 are operationally connected on an inlet side to an inlet manifold 606 where a live culture enters 605 the culture chamber 604 and on an outlet side to an outlet manifold 611 where a live culture exits 610 the culture chamber 604. One or more support pillars 620 are operationally connected to the backplate 615 and the porous material cover 660. The backplate 615 has a port for a spray distribution ball 622 for cleaning and sterilizing solutions. The negative pressure cowl 625 is operationally connected to a suction plenum for gases 626 which is operationally connected to a blower or gas fan 627 driven by a motor 628. The blower or gas fan 627 is also operationally connected to a harvest gas pressure flow control valve 630. The cowl 625, plenum 626, blower 627, and motor 628 are utilized to discharge or remove 654 stale cowl gases and simultaneously refresh 652 the gas atmosphere under the cowl 625 by sucking in fresh gas through one or more inlets 635 operationally connected to the cowl 625. The outlet manifold 611 is operationally connected to a liquid reservoir 665 into which medium 641 is delivered. The liquid reservoir 665 has an organism/cell and nutrient inlet port 672 and a liquid fill port 673. The liquid reservoir 665 provides an open space whereby liquid inventory and rise and fall depending on culture conditions. The presence of this liquid reservoir ensures that the section of the bioreactor containing the porous material cover 660 can always be 100% liquid full. The reservoir is located at the hydraulic high point of the system, and the pump 676 is at the hydraulic low point of the system. The liquid reservoir is operationally connected to the inlet duct 671 which is operationally connected to a bioreactor low point drain and drain valve 674, a recirculation pump 676, a pH probe 678, a dissolved gas probe 680, a temperature probe 682, a heat exchanger 684 for controlling culture medium 641 temperature using a cooling/heating medium 686, and a sterile air inlet 640. The inlet duct is operationally connected to the inlet manifold 606. One or more artificial sources of photoradiation 690 are optionally attached to the inside surface of the cowl 625 to provide photoradiation that can pass through the porous material cover 660. One or more sections of the cowl are optionally photopermeable 695, allowing natural or artificial photoradiation to pass through the cowl sections 695 and the porous material 660. When the bioreactor is in use, a culture within the culture medium 641 is circulated through the bioreactor. Atmospheric gases can enter the culture medium 641 by migrating in through 642 the porous material 660. The culture produces by-product gas shown as bubbles which rise through the medium 641 and migrate out 643 through the porous material cover 660. The porous material cover 660 is made of a porous material that is photopermeable, gas permeable to all gases that must be vented in and out of the bioreactor for production of the selected product, and that is of an open pore structure capable of functioning as a sterile barrier unless monoseptic conditions are otherwise maintained. All connections and seals are capable of maintaining monoseptic conditions, as necessary.

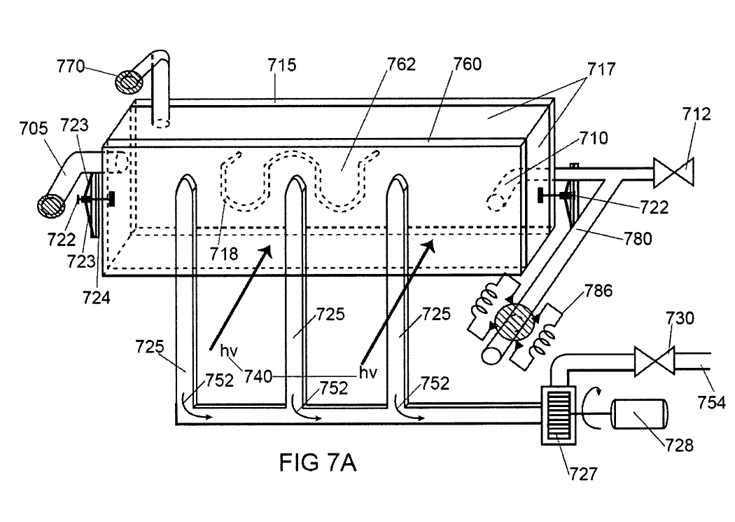

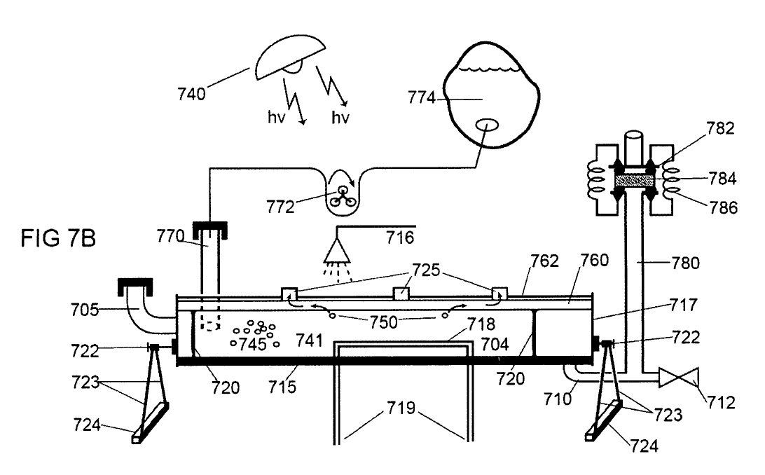

FIGS. 7A and 7B illustrate an angled overview perspective and a front cross-sectional view on a pivot stand 722, 723, 724, respectively, of a class IV bioreactor with a porous material cover 760. The bioreactor has a bottom backplate 715 operationally connected to one or more retaining walls 717. The retaining walls 717 are operationally connected to a porous material cover 760, which is operationally connected to negative pressure plenum 725 for gases. One or more support pillars 720 are operationally connected to the backplate 715 and the porous material cover 760. The porous material cover 760 outer surface area that is not in contact with the plenum is sealed with a photopermeable gas impermeable film 762. The plenum 725 is operationally connected to a squirrel cage blower 727 which is operationally connected to and driven by a motor 728. Gas traveling into the plenum is pulled towards 752 the squirrel cage blower 727 to the harvest gas pressure and flow control valve 730 for harvest gas collection 754. A retaining wall 717 of the bioreactor contains a live culture batch inlet port 705 and a nutrient addition port 770. The nutrient addition port is operationally connected to an aseptic addition transfer pump 772 and a nutrient supply bag 774. The backplate 715 contains a batch drain port 710 connected to a standpipe for accommodating volume change and providing positive pressure 780, and a batch drain port isolation valve 712. The liquid standpipe 780 functions as a reservoir. Within the standpipe 780 is a second porous material 784 held in place by a spring tension clamp 786 and sterile-sealed by a gasket 782. The walls 717 are operationally connected to a pivot stand by a pivot axis 722 which is operationally connected to support members 723 and a support plinth 724, for tilting the bioreactor culture chamber 704. The bioreactor culture chamber 704 is optionally tilted to maximize receipt of photoradiation. The backplate 715 has sterile-sealed ports for a heating/cooling internal coil 718 which allows heating/cooling media in and out 719. Medium 741 in the bioreactor can also be cooled externally by a water shower for evaporative cooling 716. The porous material cover 760 can be photopermeable to photoradiation from an optional source of photoradiation 740. When the bioreactor is in use, a culture in the medium 741 evolves gas 745 which migrates through (750) the porous material cover 760 into the plenum 725, for harvest. The porous material cover 760 is made of a porous material that is gas permeable to all gases that must exit the bioreactor and that is of an open pore structure capable of functioning as a sterile barrier. The second porous material filter 784 is made of a porous material that is gas permeable to all gases that must enter or the bioreactor and that is of an open pore structure capable of functioning as a sterile barrier in order to maintain an ambient pressure headspace above the liquid level in the standpipe. When the cultured organism/cell requires light, the porous material cover 760 is also photopermeable. All connections and seals are capable of maintaining monoseptic conditions, as necessary.

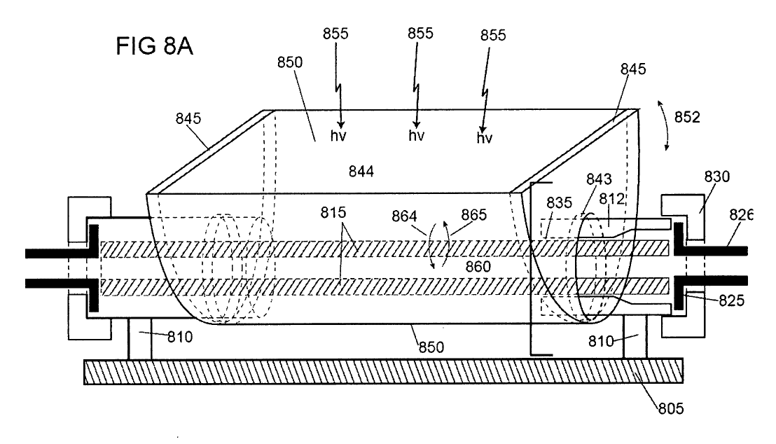

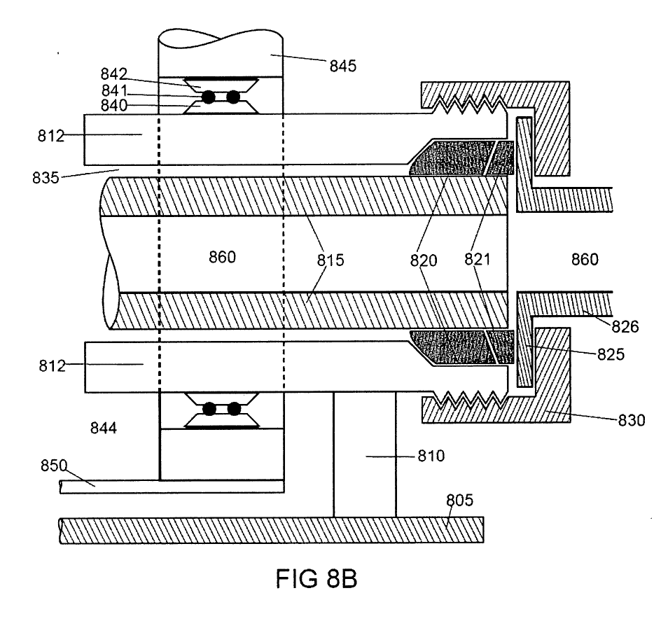

FIGS. 8A and 8B illustrate an angled side view of a class IV bioreactor with a cylindrical porous material 815 and a detail view of a connection between the porous material 815, the recirculation piping 826, and a parabolic photoradiation collector 844, respectively. A cylindrical porous material hollow along the centerline 815 is horizontally oriented and attached to recirculation piping 826 via a flange 825 and a threaded collar 830. The porous material 815 is held in place by a stationary anchoring collar (812) which in turn is supported by a vertical support pedestal 810 and a support plinth 805. A parabolic light collector 844 consists of a parabolic wall with mirrored surface 850 and parabolic end plates 845 having openings for the porous material 815. The holes in the end plates 845 are centered on the focal axis of the parabolic mirrored surface 850 from which photoradiation is reflected when a photoradiation source 855 in utilized. The parabolic light collector 844 is rotatably connected 852 to the stationary anchoring collar 812 by a ball bearing assembly 843. The ball bearing assembly consists of an inner race 840 stationarily connected to the stationary anchoring collar 812, an outer race 842 stationarily connected to the parabolic end plates and ball bearings 841. The porous material 815 is held in a fixed position within the stationary anchoring collar operationally connected to the flange 825 of the recirculation piping 826 by a larger compression ferrule 820, a smaller compression ferrule 821, a stationary anchoring collar 812, and a threaded collar 830. When the threaded collar 830 holds the stationary anchoring collar 812 tightly, the ferrules are compressed against the porous material 815 and the flange 825 of the recirculation piping 826 such that a hydraulic seal is formed, and there is an incidental air gap 835 between the porous material 815 and the stationary anchoring collar 812. When the bioreactor is in use, culture fluid 860 containing a selected culture is circulated through the porous material 815 and the recirculation piping. The connection between the porous material 815 and the recirculation piping is capable of maintaining monoseptic conditions. The porous material is gas permeable, allowing multi-directional gas exchange, e.g., CO₂ into 864 the culture fluid 860 and O₂ out 865. The porous material is also photopermeable to photoradiation received directly from the photoradiation source 855 and to photoradiation reflected off of the parabolic collector mirrored surface 850. The porous material is of an open pore structure capable of functioning as a sterile barrier. All connections and seals are capable of maintaining monoseptic conditions, as necessary.

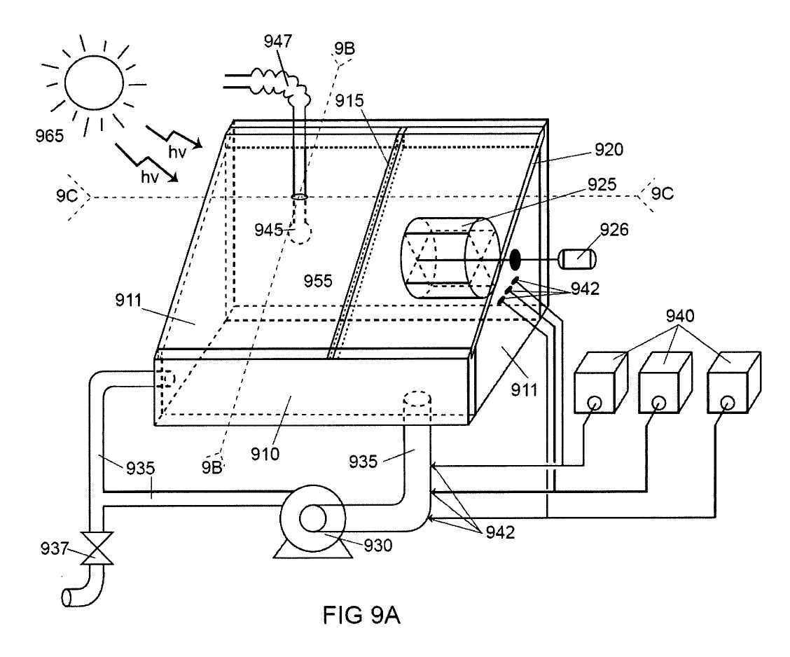

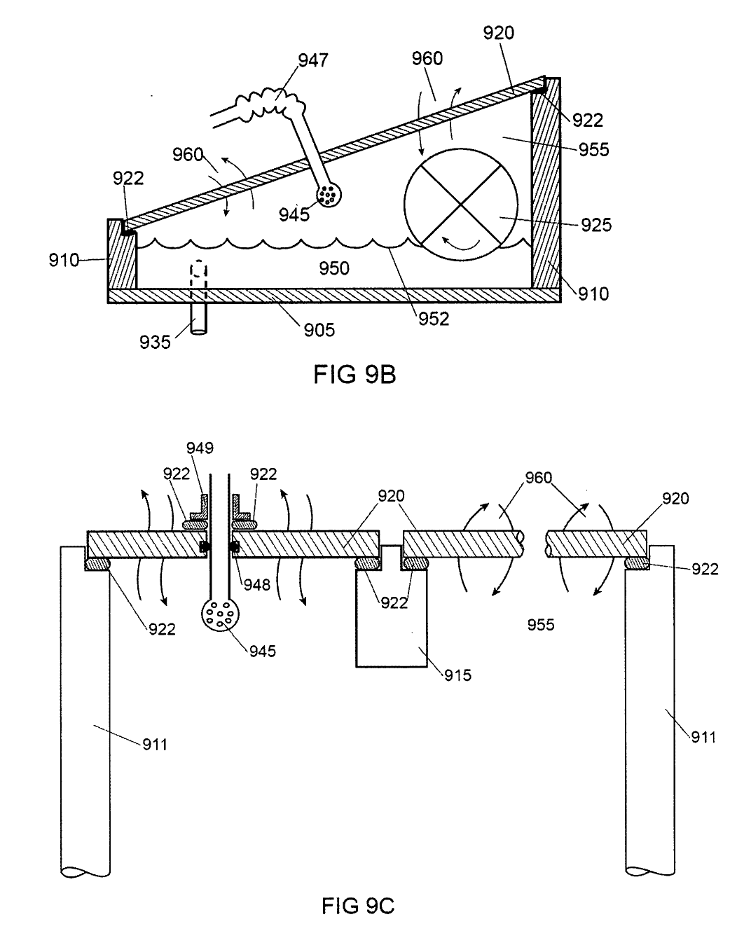

FIGS. 9A-9C illustrate an top-angled front view, a side cross-sectional view, and a front cross-section view through line A of a class V bioreactor with a porous material tent lid 920. A bottom 905 is operationally connected to sides 910 and 911 which may or may not extend vertically to and/or support the porous material. The porous material tented lid 920 is attached to at least two of the sides 910 or 911 by adhesive gaskets 922. The porous material tented lid covers gas headspace 955 inside the bioreactor. The porous material tent lid 920 is optionally supported by one or more structural support ribs 915, sealed with gaskets 922. The culture fluid 950 is at a liquid level 952 below the height of the shortest side 910 or 911. One or more sides (910 or 911) and bottom 905 have ports for recirculation piping 935. A recirculation pump 930 operationally connected to the recirculation piping 935 circulates the culture fluid 950. A system drain and harvest valve 937 is operationally connected to the recirculation piping 935. Addition ports 942 in the recirculation piping 935 and in bioreactor walls 910 and 911 are operationally connected to reservoirs for nutrient, acid/base reagents, and inoculum 940. The porous material tent lid 920 has a port for a spray distribution ball (945) for cleaning and sterilizing solutions operationally connected to a flexible connection line 947. The spray distribution ball 945 is attached to the porous material lid 920 by a structural support collar 949 holding adhesive gaskets 922, and the connection is further sealed with an O-ring 948. A paddlewheel agitator drive motor 926 is operationally connected through a port in a side (910 or 911) or above a side (910 or 911) to a paddlewheel agitator 925 that is at least partially submerged in the culture fluid 950. The porous material lid 920 is multi-directionally gas permeable 960, e.g. carbon dioxide or oxygen can migrate from the outside air into the bioreactor and oxygen or carbon dioxide from the bioreactor can migrate through to the outside air. The porous material tent lid 920 is optionally photopermeable for transmitting photoradiation from a natural or artificial source (965).

The bioreactors illustrated in the figures can be utilized as follows:

The Class I bioreactor illustrated in FIGS. 1A-1C can be used as follows: The bioreactor is cleaned, sterilized, and rinsed using the spray distribution ball 147. The bioreactor is aseptically filled with an initial growth medium suitable for growth of the production organism. The initial growth medium is circulated with the agitator, and the environmental parameters are adjusted for the selected organism. The growth medium is inoculated with a small amount of the production organism obtained from a commercial source. Gas appropriate for the selected cell/organism is sparged into the culture medium 141 using the sparge tube 130. The environmental parameters are monitored and maintained in a range suitable for growth of the production organism. Exhaust gases including gases produced by the production organism pass through the porous material lid 155 of the bioreactor. When a sufficient cell mass has been achieved in the bioreactor, the environmental conditions are optionally manipulated to promote production of the selected product. When the producing culture has produced sufficient product, the product is harvested. The bioreactor can optionally be reused.

The Class I bioreactor illustrated in FIGS. 2A-2D can be used as follows: The bioreactor is cleaned, sterilized, and rinsed. The bioreactor is aseptically filled with an initial growth medium suitable for growth of the production organism. The initial growth medium is circulated with the agitator, and the environmental parameters are adjusted for the selected organism. Introduced sparge gases migrate through the porous material dome 230. The growth medium is inoculated with a small amount of the production organism obtained from a commercial source. The environmental parameters are monitored and maintained in a range suitable for growth of the production organism. Exhaust gases including gases produced by the production organism pass through vent 260 and migrate through the porous material filter 262. When a sufficient cell mass has been achieved in the bioreactor, the environmental conditions are optionally manipulated to promote production of the selected product. When the producing culture has produced sufficient product, the product is harvested. The bioreactor can optionally be reused.

The Class II bioreactor illustrated in FIGS. 3A-3B can be used as follows: The bioreactor is cleaned, sterilized, and rinsed. The bioreactor is aseptically filled with an initial growth medium suitable for growth of the production organism. The initial growth medium is circulated, and the environmental parameters are adjusted for the selected organism. For hydrogen production by photosynthetic algae, an inert gas is introduced into the cowl using the blower or gas fan to maintain an inert gas flow sweeping across the porous material cover 360 and a negative pressure within the cowl 325. The growth medium is inoculated with a production organism obtained from a commercial source. The environmental parameters are monitored and maintained in a range suitable for growth and/or maintenance of the production organism. When a sufficient cell mass has been achieved in the bioreactor, the environmental conditions are optionally manipulated to promote production of the selected product. For hydrogen production using photosynthetic algae, the inert gas flow is reduced when the concentration of dissolved oxygen in the culture medium reaches about zero. Exhaust gases, including hydrogen gas produced by the production organism, pass through the porous material cover 360 of the bioreactor. Hydrogen gas is harvested by additional elements connected to the discharge of the blower or gas fan 327. The culture organisms are maintained for as long as possible in a hydrogen producing state as is known in the art. The bioreactor can optionally be reused.

The Class II bioreactor illustrated in FIGS. 4A-4B can be used as follows: The bioreactor is sterilized. The bioreactor is aseptically filled with an initial growth medium suitable for growth of the production organism so that the growth medium is in liquid contact with the entire bottom surface 411 of the porous material window 410. The environmental parameters are adjusted for the selected organism. The bioreactor can be placed in a temperature/pressure regulated tissue culture incubator. The growth medium is inoculated with a small amount of the production organism obtained from a commercial source. The environmental parameters are monitored and maintained in a range suitable for growth of the production organism. Gases necessary for the selected organism/cell migrate through the porous material window 410 to the culture medium. Exhaust gases including gases produced by the production organism/cell migrate from the culture medium through the porous material window 410 and out of the bioreactor. When a sufficient cell mass has been achieved in the bioreactor, the environmental conditions are optionally manipulated to promote production of the selected product. When sufficient product has been produced and harvested, the bioreactor is discarded. The bioreactor illustrated in FIGS. 4A-4B can be utilized as a Class IV bioreactor if the porous window is photopermeable and appropriate photoradiation is provided.

The Class III bioreactor illustrated in FIGS. 5A-5C can be used as follows: The bioreactor is cleaned, sterilized, and rinsed using the spray distribution ball 547. The bioreactor is aseptically filled with an initial growth medium suitable for growth of the production organism. The initial growth medium is circulated with the agitator, and the environmental parameters are adjusted for the selected organism. The growth medium is inoculated with a small amount of the production organism obtained from a commercial source. Gas appropriate for the selected cell/organism is sparged into the culture medium 541 using the sparge tube 530. The environmental parameters are monitored and maintained in a range suitable for growth of the production organism. Photoradiation permeates the porous material cover 555. Exhaust gases including gases produced by the production organism pass through the porous material cover 555 of the bioreactor. When a sufficient cell mass has been achieved in the bioreactor, the environmental conditions are optionally manipulated to promote production of the selected product. When the producing culture has produced sufficient product, the product is harvested. The bioreactor can optionally be reused.

The Class IV bioreactor illustrated in FIGS. 6A-6B can be used as follows: The bioreactor is cleaned, sterilized, and rinsed. The bioreactor is aseptically filled with an initial growth medium suitable for growth of the production organism. The initial growth medium is circulated, and the environmental parameters are adjusted for the selected organism. Fresh air is pulled under the cowl 625 above the porous material cover 660 through fresh air ducts 635. The growth medium is inoculated with a production organism obtained from a commercial source. The environmental parameters are monitored and maintained in a range suitable for growth of the production organism. Gases in the culture medium equilibrate with the gases in the air above the porous material cover 660. Optional photoradiation sources 690 provide photoradiation which permeates the porous material cover 660 to reach the culture. Optional photopermeable sections 695 of the cowl 625 allow receipt of photoradiation. When a sufficient cell mass has been achieved in the bioreactor, the environmental conditions are optionally manipulated to promote production of the selected product. When the producing culture has produced sufficient product, the product is harvested. The bioreactor can optionally be reused.

The Class IV bioreactor illustrated in FIGS. 7A-7B can be used as follows: The bioreactor is cleaned, sterilized, and rinsed. The bioreactor is aseptically filled with an initial growth medium suitable for growth of the production cell/organism. The environmental parameters are adjusted for the selected cell/organism. The growth medium is inoculated with a production organism obtained from a commercial source. The environmental parameters are monitored and maintained in a range suitable for growth of the production organism. Gases migrate through the porous material filter 784 from the air to the medium and/or from the medium to the air in order to equalize the pressure in the bioreactor with that of the external environment. Photoradiation is optionally provided. Photoradiation from a natural or an artificial source permeates the porous material cover 760. The bioreactor culture chamber 704 is optionally tilted to optimize photoradiation received. Gases from the cells/organism in the medium migrate through the porous material cover 760 to the plenum 725 where they are optionally harvested. For hydrogen production, the culture organisms are maintained in a hydrogen producing state as is known in the art. The bioreactor can optionally be reused.

The Class IV bioreactor illustrated in FIGS. 8A-8B can be used as follows: The bioreactor is cleaned, sterilized, and rinsed. The bioreactor is aseptically filled with an initial growth medium suitable for growth of the production cell/organism. The initial growth medium is circulated, and the environmental parameters are adjusted for the selected organism. The growth medium is inoculated with a production organism obtained from a commercial source. The parabolic photoradiation collector 844 is oriented to optimize photoradiation received from an artificial or natural photoradiation source. The environmental parameters are monitored and maintained in a range suitable for growth of the production organism. Gases migrate through the porous material 815 from the air to the medium and/or from the medium to the air. The parabolic collector 844 is reoriented as necessary. When the producing culture has produced sufficient product, the product is harvested. The bioreactor can optionally be reused.

The Class V bioreactor illustrated in FIGS. 9A-9C can be used as follows: The bioreactor is cleaned and optionally sterilized. The bioreactor is aseptically filled with an initial growth medium suitable for growth of the production cell/organism. The initial growth medium is circulated using a recirculation pump 930 and/or a paddlewheel 925, and the environmental parameters are adjusted for the selected organism. The growth medium is inoculated with a production organism obtained from a commercial source. Photoradiation is provided by a natural or an artificial source. Air flows freely into and out of the bioreactor through gaps between the porous material tent lid 920 and the sides 910 and 911 and/or gases migrate through the porous material tent lid 920. The environmental parameters are monitored and maintained in a range suitable for growth of the production organism. When the producing culture has produced sufficient product, the product is harvested. The bioreactor can optionally be reused.

This invention provides bioreactors comprising selectively permeable porous materials having open pore structures. Porous materials useful in the practice of this invention are selectively permeable to heat, electricity, sound, gases, photoradiation, visible radiation, ultraviolet radiation, cells, organisms, cellular components, and other unwanted contaminants. The bioreactors provided by this invention do not utilize the porous material only as a support network for a cell, organism, or cellular component to be cultured by the bioreactors. Porous materials useful in the practice of this invention have an open pore structure and are optionally gas permeable, photopermeable, transparent, hydrophobic, capable of functioning as a sterile barrier, capable of being made in a wide variety of forms, and/or recyclable. In an embodiment of this invention, a bioreactor comprises a porous material having an open pore structure that is gas permeable. In an embodiment of this invention, a bioreactor comprises a porous material having an open pore structure that is gas permeable and photopermeable. In an embodiment of this invention, a bioreactor comprises a porous material having an open pore structure that is gas permeable, photopermeable, and a sterile barrier. In an embodiment of this invention, a bioreactor comprises a porous material having an open pore structure that is gas permeable and a sterile barrier. In an embodiment of this invention, a bioreactor comprises a porous material having an open pore structure that is gas permeable and hydrophobic.

Porous materials useful in the practice of this invention include, but are not limited to, aerogels, xerogels, and sol-gel glasses. In an embodiment of this invention, a bioreactor comprises a porous material having an open pore structure that is an aerogel, xerogel, or a sol-gel glass. Aerogels useful in the practice of this invention include hydrophobic aerogels and silica aerogels. In an embodiment of this invention, a bioreactor comprises a porous material having an open pore structure that is a hydrophobic aerogel or a silica aerogel. Aerogels that are not hydrophobic are not useful in the practice of this invention. Aerogels useful in the practice of this invention are not hydrophobic-liquid permeable. When an aerogel of this invention is in liquid contact with a culture medium, the culture medium does not permeate the aerogel. Methods that compromise a structural or hydrophobic property of an aerogel in a bioreactor of this invention, are not useful with the bioreactors of this invention. Methods that destroy the hydrophobicity of an aerogel in a bioreactor of this invention are not useful in the practice of this invention when the bioreactor comprises an aerogel. High pressure steam methods (e.g., for sterilization) are not useful in the practice of this invention when the bioreactor comprises an aerogel that is structurally or functionally compromised by the high pressure steam.

Porous materials are useful in all locations and functions in which materials that are gas permeable, photopermeable, transparent, or capable of functioning as a sterile barrier, are useful in bioreactors, wherein the required structural and functional properties of the porous materials are not compromised. Porous materials are particularly useful as structural and/or functional components of bioreactors because of their structural properties including low density, light weight, strength and rigidity.

This invention provides bioreactors comprising porous materials as one or more portions of or entire structural or functional components of the bioreactors, including, but not limited to, walls, covers, floors, filters, windows, and tubes. In an embodiment of this invention, a bioreactor comprises a porous material having an open pore structure that functions as a wall, cover, floor, window, and/or filter. Bioreactors provided by this invention include porous materials that permit fluid communication between the contents (gaseous or liquid) of the bioreactor and the atmosphere outside the bioreactor (the earth's atmosphere or a controlled atmosphere). Bioreactors provided by this invention comprise porous material forms including panels, monoliths, cylindrical vessels, cylindrical tubes, hemispheres, and portions or combinations thereof. This invention provides bioreactors comprising more than one porous material. This invention provides bioreactors having transparent and photopermeable porous materials functioning as windows. Porous windows are useful in all locations that non-porous material windows are useful in bioreactors, including walls, covers, cowls, and viewing ports. When a porous material is utilized in a bioreactor of this invention, sufficient structural supports, e.g., braces, pillars, ribs, are utilized, to enable the porous material to perform its selected function(s). Methods for determining sufficient structural support are known in the bioreactor, engineering, and porous material arts. Factors to be considered include the physical properties of the porous material, the size and weight of the bioreactor both filled and empty, and the physical orientation of the porous material. Sealing methods and materials that spread a compressive load over a larger surface area of the porous material are preferred in the practice of this invention. Compression ferrules and flat gaskets that distribute a compressive load are preferred over O-ring seals. In an embodiment of this invention, a bioreactor comprises a porous material having an open pore structure is sealed using compression ferrules, flat gaskets, or O-rings.

Bioreactors of this invention utilize porous material filters at various locations and for various functions within the bioreactor. Separate and distinct porous filters are useful to filter incoming and outgoing gases even when the primary function of the outgoing gas filter is to prevent contamination of the culture fluid (see Example 2 and FIGS. 2A-2D). The primary purpose of an outgoing gas filter is to prevent contamination. One porous cover can be used to filter incoming or outgoing gases (Example 7 and FIGS. 7A-7B). One porous material can simultaneously function as a wall or cover and optionally be a filter, such as a sterile barrier filter (Examples 1, 3-6, and 8). In an embodiment of this invention, a bioreactor comprises porous material filters having an open pore structure that are utilized to filter incoming gas, outgoing gas, both incoming and outgoing gas, and/or cells, organisms and/or cellular components.

Gas permeable porous materials useful in the practice of this invention are optionally also selectively gas permeable, and simultaneously photopermeable and/or transparent. Gas permeable porous materials are optionally permeable to acetylene, air, ammonia, argon, bromine, carbon dioxide (CO₂), carbon monoxide (CO), chlorine, ethane, ethylene, ethylene oxide, formaldehyde, helium, hydrogen, hydrogen chloride, hydrogen cyanide (HCN), hydrogen iodide, hydrogen sulfide, methane, methyl chloride, nitric oxide (NO), nitrogen, nitrous oxide (N₂O), oxygen, sulfur dioxide, gaseous fluorocarbons, sulfur dioxide, and volatile organic molecules or selected subsets of these gases. In an embodiment of this invention, a bioreactor comprises a porous material having an open pore structure that is gas permeable to all gases that are required to enter and exit the bioreactor for production of the selected product.

When designing a bioreactor of this invention, the required functions of the porous material are considered and a porous material (e.g., composition and form) is selected that is capable of performing those functions. A selected porous material can have additional properties that do not interfere with the porous material performing the required functions or interfere with the functioning of the bioreactor. For example, a porous cover for an open pond style bioreactor that is not maintained in a monoseptic condition, can be capable of functioning as a sterile barrier despite that this characteristic is not utilized in this bioreactor. Similarly, a porous material that is utilized for gas permeability can also be photopermeable, despite that no photoradiation is required to migrate through it, providing that if photoradiation is present and does migrate through it that this does not interfere with the functioning of the bioreactor for the selected culture. Also, a porous window that is required to be translucent, can also be photopermeable and/or gas permeable, providing that the photopermeable and gas permeable properties do not interfere with the desired functioning of the bioreactor.

This invention provides bioreactors optionally also having a means for one or more of the following processes: bioreactor sanitization and/or sterilization; preventing contamination; culture agitation or circulation; temperature detection and/or control; gas delivery and/or removal (e.g., degassing); dissolved gas detection and/or control; pH detection and/or control; photoradiation delivery; detecting and/or quantitating photoradiation reception; reorienting a portion of the bioreactor relative to a source of photoradiation; liquid delivery and/or removal; nutrient detection and/or delivery; waste removal; cell and organism delivery and/or removal; gas harvesting; harvesting product; monoseptic holding or storing of culture fluids; monoseptic processing of culture fluids where gas exchange is required; and monoseptic holding or storing of culture fluids during operations designed to concentrate, purify, separate, isolate or otherwise treat culture fluids in the process of producing a purified product.

This invention provides bioreactors having porous materials having a pore size of about 1000 nm, 500 nm, or about 200 nm. This invention provides bioreactors having porous materials having a pore size small enough that the porous material functions as a sterile barrier. This invention provides bioreactors having porous materials having pores having diameters greater than about 0.1 nm or about 0.5 nm. This invention provides bioreactors having porous materials having pore diameters between about 0.1 nm and about 1000 nm, between about 1 nm and about 500 nm, between about 2 nm and about 250 nm, between about 3 nm and about 100 nm, or between about 5 nm and about 50 nm. Methods are known in the art for making porous materials with a selected range of pore diameters and/or with a selected pore size.

This invention provides bioreactors comprising porous materials wherein the primary function of the porous materials is not to embed or be a support network for a cell, organism, or cellular component. This invention provides bioreactors comprising porous materials having a function other than to embed or be a support network for a cell, organism, or cellular component. Porous materials useful in the practice of this invention can, during bioreactor operation, contain one or more cells, organisms, or cellular components within the pore structure providing that the cells, organisms, or cellular components are a minority, or are not a substantial proportion of those to be cultured by the bioreactor. Porous materials useful in the practice of this invention can during bioreactor operation contain one or more cells, organisms, or cellular components in the open pore structure providing that the cells, organisms, or cellular components are only in the open pore structure transiently, and wherein the cells, organisms, or cellular components are not immobilized within the porous material.

This invention provides bioreactors comprising porous materials made by a sol-gel process.

Bioreactors provided by this invention are useful for separating gaseous products from the culture medium.

This invention provides a method for making a bioreactor of this invention comprising: providing a porous material; providing remaining bioreactor components; and assembling the porous material and the components.

This invention provides a method for culturing a cell comprising: providing a bioreactor of this invention; providing a cell; and providing environmental conditions whereby the cell is cultured. The bioreactors of this invention are capable of culturing cells that are viral, bacterial, animal, plant, algal, or fungal cells, including insect and mammalian cells. This invention provides a method for culturing a cell of Chlamydomonas reinhardtii, Chlamydomonas moewusii, Chlamydomonas sp. strain MGA161, Chlamydomonas eugametos, and Chlamydomonas segnis belonging to Chlamydomonas; Chlorella vulgaris belonging to Chlorella; Senedesmus obliguus belonging to Senedesmus; and Dunaliella tertrolecta belonging to Dunaliella, Anabaena variabilis ATCC 29413 belonging to Anabaena, Cyanothece sp. ATCC 51142 belonging to Cyanothece, Synechococcus sp. PCC 7942 belonging to Synechococcus, Anacystis nidulans belonging to Anacystis, Rhodopseudomonas palustris and Rhodopseudomonas acidophila belonging to Rhodopseudomonas, and Rhodospirillum rubrum ATCC 11170, Rhodospirillum rubrum IFO 3986 belonging to Rhodospirillum, Rhodobacter sphaeroides, Rhodobacter capsulatus ATCC 23782, ATCC 17013 belonging to Rhodobacter, and Rhodovulum strictum, Rhodovulum adriaticum, Rhodovulum sulfidophilum belonging to Rhodovulum, purple nonsulfur bacteria belonging to Rhodospirillaceae, or green gliding bacteria belonging to Chloflexaceae.

This invention provides a method for culturing a cell that produces a product, wherein products are not limited to, but include hydrogen gas, biomass, chemicals, and pharmaceuticals. This invention provides a method for culturing an organism comprising: providing a bioreactor of this invention; providing an organism; providing nutrients for the organism; adding the nutrients and the organism to the bioreactor; and providing environmental conditions whereby the organism is cultured. Environmental conditions useful for culturing cells, organism, and cellular components are known in the art. The method for culturing organisms is useful for culturing plants and/or animals. The bioreactors of this invention are useful for culturing as yet to be discovered cells, organisms, cellular components, including using as yet to be discovered methods, including for performing as yet to be discovered biochemical pathways, and for producing as yet to be discovered products.

This invention provides a method for producing hydrogen gas comprising: providing a bioreactor of this invention; providing a hydrogen-producing cell or organism; providing environmental conditions; and allowing the cell or organism to produce hydrogen. This invention provides a method for producing a product selected from the group consisting of gaseous products, biomass, chemicals, and pharmaceuticals, the method comprising: providing a bioreactor of claim 1; providing a cell, organism, or cellular component capable of producing the product; providing environmental conditions whereby the cell, organism, or cellular component produces the product; and allowing the cell or organism to produce the product.

All bioreactor styles, configurations, and sizes known in the art are useful in the practice of this invention. Bioreactor sizes include microscopic bioreactors, hand-held bioreactors, laboratory size bioreactors, and industrial production scale bioreactors. This invention provides reusable bioreactors, single-use bioreactors, and presterilized bioreactors requiring no user sterilization prior to use.

The bioreactors of this invention are useful for culturing methods with and without requirements for photoradiation. When utilizing cells, organisms, cellular components, or methods requiring photoradiation, the bioreactor comprises a means for producing, receiving, and/or delivering photoradiation to the culture. Photoradiation sources useful in the practice of this invention include artificial and natural photoradiation sources. Examples 4-5, and 7-10 describe bioreactors with external photoradiation or light sources that utilize photopermeable and/or transparent porous materials. Example 6 describes a bioreactor with an internal photoradiation source that utilizes a photopermeable porous material as a sterile barrier between the photoradiation source in the cowl attached to the ductwork.

The bioreactors of this invention optionally utilize porous materials as sterile barriers. This invention provides bioreactors having porous materials useful for decreasing, but not preventing contamination. FIGS. 9A-9C illustrate an open pond style bioreactor in which the porous material can be used as a cover to decrease the amount of contaminating material that is likely to enter the open pond. This same cover for a similar style bioreactor may optionally prevent contaminating material from entering (i.e., function as a sterile barrier) if the porous material, walls and other bioreactor components are operationally connected to completely separate the culture fluid from the external environment. This invention also provides bioreactor covers comprising a porous material. A bioreactor cover for an open pond style bioreactor, need not be part of the bioreactor, but can be a separate object, as in Example 12.

Bioreactors comprising an open pore structured porous material, including aerogels, are useful for microorganism, mammalian, plant, and insect cell culture production of metabolites including pharmaceuticals and other chemicals. Both intracellular and extracellular products can be produced using bioreactors of this invention. All bioreactor designs known in the art, or yet to be invented, can be built with a porous material wall or lid, provided that sufficient structural support is provided for the porous material. Preferably, the bioreactors of this invention are useful for production of products, including but not limited to hydrogen, chemicals, biomass, and/or pharmaceuticals.

Open pore structured porous materials useful in the practice of this invention include materials comprising one or more inorganic solids such as an oxide, meso-porous silicates, and layered materials. When photopermeable porous materials are desired, the porous material is preferably produced with a silica containing precursor. Other oxides of metals or non-metals such as aluminum, titanium, zirconium, and mixtures thereof, are precursors useful for making porous materials useful in the practice of this invention. Useful silica containing precursors include tetramethylorthosilicate (TMOS) and TEOS.

Porous materials useful in the practice of this invention are optionally selectively permeable, including completely permeable and completely a barrier, to gases, photoradiation, visible radiation, ultraviolet radiation, hydrophobic liquids, hydrophilic liquids, cells, organisms, cellular components, and/or unwanted contaminants. Porous materials useful in the practice of this invention that are selectively permeable to radiation include porous materials permeable to photoradiation, porous materials not permeable to photoradiation, and porous materials that are permeable to photoradiation for some selected organisms/cells but not others. Porous materials of this invention are selectively permeable as a result of the open pore structure, the pore size, and/or the characteristics of the material that is not the pores (i.e., the solid component of the material).

The bioreactors of this invention are operable with all ranges of shear from no shear through low shear up to and including sufficient recirculation flow rates to keep unicellular organisms in suspension. The bioreactors of this invention are also operable with high shear rates typically required for mass transfer of gases such as oxygen from sparged bubbles to the culture medium.

When making a porous material for a bioreactor of this invention, the style of bioreactor and function of the porous material are considered when selecting a method for making, the recipe of, and the final form of the porous material.

Of the bioreactors provided by this invention, Class I and II bioreactors are particularly appropriate for culturing heterotrophs and chemotrophs, and Classes III and IV are particularly appropriate for culturing phototrophs or photosynthetic cells and organisms.

This invention provides vessels useful for culturing a cell, organism, or cellular component wherein the vessel comprises a porous material which functions in the culturing process.

Bioreactors provided by this invention are optionally not useful for culturing humans and/or other selected organism or cell. Bioreactors provided by this invention are optionally not a vessel for human habitation.

Methods are known in the art for making aerogels. All known methods for making hydrophobic aerogels known in the art are useful in the practice of this invention, including, but not limited to, methods described in “Sol Gel Processing of Ceramics and Glass,” Market Report, July 2002, Business Communications Co, Norwalk, Conn., USA. Methods are known in the art for making aerogels that are photopermeable, gas permeable, and/or hydrophobic. Example 11 describes a method for making an aerogel useful in the practice of this invention.

Bioreactors of this invention can be used to make a variety of products: biomass, chemical, pharmaceutical, and gaseous products.

Hydrogen represents an ideal source for a clean, renewable energy. Anaerobic bacteria and photosynthetic microorganisms such as photosynthetic bacteria, cyanobacteria, and algae are useful for producing hydrogen. Hydrogen is produced by biophotolysis, requiring light, or from organic substrates, such as wastes. The photosynthetic biochemistry of hydrogen production is well known (Akkerman (2002) Intl J of Hydrogen Energy 27:1195-1208; Hallenbeck (2002) Intl J of Hydrogen Energy 27:1185-1193).

Photosynthetic (green) algae and cyanobacteria can produce hydrogen in the absence of light and under anaerobic conditions by oxidizing carbohydrates to produce organic molecules (as has been demonstrated in Chlamydomonas), by oxidizing organic molecules (denote as CHO, meaning, for example, organic acids and aldehydes, alcohols and esters) to produce CO₂ (as has been demonstrated in cyanobacteria) and by oxidizing water (as has been demonstrated in cyanobacteria). These processes are referred to generally as biophotolysis and employ the well-studied hydrogenase enzymes in cyanobacteria. Hydrogenase enzymes are widespread in the microbial world, having been identified in anaerobic bacteria, aerobic eukaryotes including Arabidopsis, and many (but not all) green algae including Chlamydomonas, Chlorococcum, Chlorella and Scenedesmus. In all the cases above, prior to hydrogen production, green algae and cyanobacteria cultures are grown up and maintained under photosynthetic conditions. These organisms employ the PSII pathway (CO₂+H₂O yields reduced carbon+O₂) to produce biomass and/or otherwise reduce CO₂ to carbohydrates and organic molecules using light.

Photosynthetic (green) algae (for example, Scenedesmus) and so-called purple bacteria (those bacteria the can employ H₂S instead of water as the reductant e.g., Rhodovulum and Rhodobacter) as well as certain heterocystous cyanobacteria (employing a light-dependent nitrogenase system) can produce hydrogen under photoradiation and anaerobic conditions by oxidizing more reduced forms of carbon (CH₂O, carbohydrates) to CO₂ and/or other organic molecules (CHO). These processes are referred to generally as photofermentation. In addition, the so-called purple bacteria can produce hydrogen in the absence of light by oxidizing CO (carbon monoxide) with water as the electron donor (the classical water gas shift reaction).

Additionally, chemotrophic anaerobic bacteria such as Enterobacter and Clostridium can produce hydrogen by the degradation of various carbon sources usually coupled with NADH/NAD+ or Fd/FdH₂ energy transferring reactions that drive hydrogen production. However, it is impossible to completely degrade carbohydrates to CO₂ and hydrogen through anaerobic fermentation because other organic molecules (CHO) are the final electron acceptors in these aerobic pathways. For this reason, if high hydrogen yields are desired, anaerobic fermentation systems employing chemotrophic bacteria are often coupled with systems employing photosynthetic organisms such as green algae, cyanobacteria or purple bacteria, where CO₂, or even oxygen itself, can be the final electron acceptor.

Methods known in the art for producing hydrogen using green algae, such as Chlamydomonas reinhardtii include those described in Kosuourov et al., National Biomass Coordination Office, U.S. Department of Energy, BCOTA, Abstract 24 Z336; Ghirardi, Proceedings of the 2001 US DOE Hydrogen Program Review, NREL/CP-570-30535 “Cyclic Photobiological Algal H₂-Production.” U.S. Patent Application Publication No. 2001/0053543 (published Dec. 20, 2001) describes a reversible physiological process for the temporal separation of oxygen evolution and hydrogen production in a microorganism, such as Chlamydomonas, comprising depleting a nutrient from the medium. U.S. Pat. No. 4,532,210 (issued Jul. 30, 1985) describes producing hydrogen by alga in an alternating light/dark cycle. Melis (2002) Intl J of Hydrogen Energy 27:1217-1228 describes hydrogen production by green alga by removing oxygen.

Methods are also known in the art for producing hydrogen using the bacteria Rubrivivax gelatinousus CBS and CBS2 (Wolfrum, Proceedings of the 2002 US DOE Hydrogen Program Review, NREL/CP-610-32405 “Bioreactor Development for Biological Hydrogen Production”; Wolfrum, Proceedings of the 2001 US DOE Hydrogen Program Review, NREL/CP-570-30535 “Bioreactor Design Studies for a Novel Hydrogen-Producing Bacterium”).

Biomass products include whole cells that are used for nutrition and to balance the flora and fauna within the digestive tract of humans, whole cells that are used for inoculating other bioreactors including those bioreactors that produce hydrogen, specialty chemicals, pharmaceuticals and alcoholic beverages, and whole cells that are used for inoculating various other biomass heaps including silage and compost piles and used to inoculate various doughs prior to baking wherein carbon dioxide production is desired as baking proceeds. Production of biomass in bioreactors is well known in the art. Production of biomass proceeds with the inoculation of the bioreactor and maintaining an environment suitable for growth which typically includes control of temperature, pH and mixing. Biomass increases in the bioreactor beyond the levels introduced in the form of the inoculum by cell division and can eventually be limited by the availability of nutrients, availability of light (in photosynthesis), accumulation of metabolic waste products, or inability to adequately mix the contents of the vessel. Biomass production within the bioreactor can also proceed indefinitely if nutrients are continuously provided, waste products are continuously removed and biomass is continuously removed to prevent it from physically accumulating in the bioreactor and interfering with agitation, gas transfer, phototransmission or some other aspect of environmental control. Biomass is harvested from the vessel either in a single batch or continuously.

A variety of chemicals can be made in or by using the bioreactors of this invention, employing microorganisms or other cells. Some methods require photoradiation, but most do not. Chemicals produced using bioreactors include, among many others, amino acids, organic monomers, vitamins, pigments and colorants used in a variety of applications; polymers such as polylactic acid, polysaccharides, polyhydroxyalkanoates and other organic polymers used in the fabrication of various plastic articles; food additives employed to affect flavor, texture, shelf life and other properties of foods; and metabolites or metabolic byproducts from the growth or maintenance of cells that has utility as a chemical reagent or raw material in the synthesis of other chemical entities. Production of chemicals using bioreactors is well known in the art. Production of chemicals proceeds with the inoculation of the bioreactor and maintaining an environment suitable for growth which typically includes control of temperature, pH and mixing. Specialty chemicals are produced either in tandem with the growth of the organisms by replicative division or as a consequence of maintaining proper environmental conditions in a culture that is no longer actively growing. The physiological state of the organism, the presence or absence of environmental factors such as high pH, low pH, high temperature, low temperature, sufficient nutrient levels, depleted nutrient levels, illumination or absence of illumination all contribute to the production of specialty chemicals by the organism present in the bioreactor. Culture fluid containing biomass, specialty chemicals, unused nutrients, metabolic waste products, dissolved gases and other suspended and/or dissolved solids, liquid and gases is removed from the bioreactor either in a single batch or continuously and subjected to further processing. Further processing includes separation of the biomass and unwanted nutrients and other suspended and/or dissolved solids, liquid and gases from the desired chemical.

A variety of pharmaceutical products can be made in the bioreactors of this invention employing microorganism or other cells. Pharmaceutical products include, among many others, antibiotics, therapeutic proteins, monoclonal antibodies, growth factors, hormones, co-factors and vaccines. Production of pharmaceutical products requires strict control of process conditions including monoseptic operation of the bioreactor. Pharmaceutical products are manufactured using both traditional microorganisms including bacteria, yeasts, molds and fungi and also mammalian, plant, and insect tissue cells. Some of the microorganisms and cells have been prepared using recombinant genetic methods and thereby contain genetic material not initially present in the cell or contain alterations of the original genetic material that enhances production of the desired pharmaceutical product by one or several possible mechanisms. Pharmaceutical products can include any metabolite or metabolic byproduct from the growth or maintenance of cells that has utility in the prevention, treatment, mitigation, diagnosis or cure of any physiological condition. Production of pharmaceutical products by microorganisms using bioreactors is well known in the art. Production of pharmaceutical products proceeds with the inoculation of the bioreactor and maintenance of an environment suitable for growth which typically includes control of temperature, pH and mixing. Pharmaceutical products are produced either in tandem with the growth of the organisms by replicative division or as a consequence of maintaining proper environmental conditions in a culture that is no longer actively growing. The physiological state of the organism, the presence or absence of environmental factors such as high pH, low pH, high temperature, low temperature, sufficient nutrient levels, depleted nutrient levels, illumination or absence of illumination, the addition of inducers all contribute to the production of pharmaceutical products by the organism present in the bioreactor. Culture fluid containing biomass, pharmaceutical products, unused nutrients, metabolic waste products, dissolved gases and other suspended and/or dissolved solids, liquid and gases is removed from the bioreactor either in a single batch or continuously and subjected to further processing. Further processing includes separation of the biomass and unwanted nutrients and other suspended and/or dissolved solids, liquid and gases from the desired pharmaceutical product.

All known bioreactor types and methods for using bioreactors known in the art are useful in the practice of this invention, including, but not limited to, bioreactors and methods described in U.S. Pat. No. 5,763,279 (issued Jun. 9, 1998), U.S. Pat. No. 6,228,607 (issued may 8, 2001), U.S. Pat. No. 6,432,698 (issued Aug. 13, 2002), UK Patent application GB 2118572, Gordon (2002) Intl J of Hydrogen Energy 27:1175-1184, BioHydrogen (1998) Plenum Press, NY, Ed Zaborsky, WO 02/31101 (filed on Oct. 10, 2001), EP 0 100 660 (filed on Jul. 29, 1983), JP 6000494 (published Jan. 11, 1994), Liang et al. (2002) Intl J of Hydrogen Energy 27:1157-165, OptiCell™, BioCrystal Ltd., Westerville, Ohio, WO 89/11529 (filed May 19, 1989), U.S. Pat. No. 6,492,149 (issued Dec. 10, 2002), EP 0 391 590 (filed on march 27, 1990), Bioreactor system design/edited by Juan A. Asenjo, José C. Merchuk, Publisher New York: M. Dekker, c1995, van't Riet, Klaas Basic bioreactor design/Klaas van't Riet, Johannes Tramper Publisher New York: M. Dekker, c1991, and McDuffie, Norton G Bioreactor design fundamentals/Norton G. McDuffie Publisher Boston: Butterworth-Heinemann, c1991.

All methods known in the art for producing hydrogen are useful in the practice of this invention, including, but not limited to methods described in U.S. Patent Application Publication No. 2001/0053543 (published Dec. 20, 2001), U.S. Pat. No. 4,532,210 (issued Jul. 30, 1985), U.S. Pat. No. 4,442,211 (issued), U.S. Pat. No. 6,395,521 (issued May 28, 2002), Wolfrum, Proceedings of the 2002 US DOE Hydrogen Program Review, NREL/CP-610-32405, Wolfrum, Proceedings of the 2001 US DOE Hydrogen Program Review, NREL/CP-570-30535, Wolfrum (2002) Applied Biotechnology and Bioengineering 98-100:611-625, Ghirardi, Proceedings of the 2001 US DOE Hydrogen Program Review, NREL/CP-570-30535, Melis (2002) Intl J of Hydrogen Energy 27:1217-1228, Teplyakov (2002) Intl J of Hydrogen Energy 27:1149-1155, Hoekema (2002) Intl J of Hydrogen Energy 27:1331-1338, (Akkerman (2002) Intl J of Hydrogen Energy 27:1195-1208, and Hallenbeck (2002) Intl J of Hydrogen Energy 27:1185-1193).

All methods known in the art for producing biomass are useful in the practice of this invention, including, but not limited to methods described in U.S. Pat. No. 5,137,828 (issued Aug. 11, 1992), and WO 96/21723 (filed on Dec. 20, 1995). Methods for producing biomass, chemicals, and pharmaceuticals can include preparing a sterile or at least sanitary vessel (bioreactor) containing an appropriate growth medium that includes a source of carbon for fermentation in the form of carbohydrate, fats, oils, organic acids and other partially reduced forms of carbon, a source of nitrogen in the form of partially digested protein, ammonium salts or urea, a source of dissolved oxygen in the case of fermentative organisms, a source of dissolved carbon dioxide and light in the case of photosynthetic organisms and sources of micro and trace nutrients that include salts of phosphorous, magnesium, iron, sulfur, boron, molybdenum and cobalt, to name a few. The bioreactor is agitated in some way to ensure complete bulk mixing of the medium and to either facilitate dissolution of gases such as oxygen required for metabolism or to facilitate dissolution of gases such as carbon dioxide for photosynthesis.

Methods are known in the art for selecting appropriate cells and/or organisms for hydrogen production, for chemical and pharmaceutical production and for biomass production, and for selecting appropriate culture environmental conditions, including nutrient, culture medium, and photoradiation needs. Methods are known in the art for harvesting products produced using bioreactors.

When a group is disclosed herein, it is understood that all individual members of the group and all subgroups, thereof, are disclosed separately. When a Markush group or other grouping is used herein, all individual members of the group and all combinations and subcombinations possible of the group are intended to be individually included in the disclosure. Whenever a range is given in the specification, for example, a temperature range, a time range, or a composition range, all intermediate ranges and subranges, as well as all individual values included in the ranges given are intended to be included in the disclosure. Every bioreactor configuration or combination of components described or exemplified, herein, can be used to practice the invention, unless otherwise stated.

In general the terms and phrases used herein have their art-recognized meaning, which can be found by reference to standard texts, journal references and contexts known to those skilled in the art. The definitions herein are provided to clarify their specific use in the context of the invention. The terms and expressions which have been employed herein are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, can be exchanged with “consisting essentially of” or “consisting of”. The invention illustratively described herein suitably may be practiced in the absence of any element or elements, limitation or limitations which is not specifically disclosed herein. In each instance herein any of the terms “comprising”, “consisting essentially of” and “consisting of” may be replaced with either of the other two terms.

One of ordinary skill in the art will appreciate that methods, device configurations and combinations, device elements, processes, organisms, cells, and media other than those specifically exemplified can be employed in the practice of the invention without resort to undue experimentation. It will be particularly appreciated by those of ordinary skill in the art that bioreactor styles, bioreactor wall locations, bioreactor cover designs, bioreactor filter locations, sealing methods, porous materials, xerogels, aerogels, sol-gel glasses, cells, organisms, cellular components, products, hydrogen production methods, hydrogen producing cells and organisms, culturing methods, culture media, inert gases, gases, culture circulation methods, bioreactor sanitizing and sterilizing methods, and gas sparge methods, other than those specifically disclosed herein are available in the art and can be readily employed in the practice of this invention All art-known functional equivalents of any such device element and combinations, methods, materials as well as cells and organisms are intended to be encompassed within the scope of this invention.

All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

One skilled in the art will readily appreciate that the present invention is well adapted to carry out the objects and obtain the ends and advantages mentioned, as well as those inherent therein. The devices, device elements, methods and materials described herein as presently representative of preferred embodiments are exemplary and are not intended as limitations on the scope of the invention. Changes therein and other uses will occur to those skilled in the art and are intended to be encompassed within this invention.

Although the description herein contains many specificities, these should not be construed as limiting the scope of the invention, but as merely providing illustrations of some of the embodiments of the invention. Thus, additional embodiments are within the scope of the invention and within the following claims. All references cited herein are hereby incorporated by reference to the extent that there is no inconsistency with the disclosure of this specification. Some references provided herein are incorporated by reference herein to provide details concerning additional starting bioreactor designs and configurations, bioreactors processes, media and conditions for use in bioreactor processes, materials, methods of analysis, and other uses of the bioreactors of the invention.

### EXAMPLES

### Example 1

A Class I bioreactor is designed and assembled that is similar to the bioreactor illustrated in FIGS. 1A-1C with a dome, lid or cover made of porous material prepared by a sol-gel process. The porous material is in a flat configuration. Gasket materials are employed to seal the porous material. This bioreactor is utilized for aseptic fermentation for the production of a pharmaceutical. The bioreactor illustrated in FIGS. 1A-1C is also useful for batch production of primary and secondary metabolites or engineered proteins using submerged culture fermentation. The porous material employed in this exemplary bioreactor simultaneously allows migration of gases out of the reactor and prevents accidental influx of contaminating organisms or outflow of production microorganisms. The use of the porous material improves and extends the bioreactor by eliminating the need for a separate gas vent that also needs to be configured as a sterile barrier.

The bioreactor is cleaned and sanitized by spraying cleaning and sanitizing chemicals and solutions, inside the reactor in such a way that the sanitizing spray comes into contact with all the inside surfaces of the bioreactor. Spray techniques to ensure complete coverage of all inside surfaces are well known in the industry and employ specially designed spray devices and distribution balls 147 in common use for similar cleaning applications. Cleaning solution drains from the bioreactor through a drain port and drain valve 107 and is either discarded or reused. Spraying and circulating continues for a sufficient time and at a sufficient temperature to effectively sanitize the inside of the bioreactor.

The bioreactor is then rinsed with clean water using the same spray devices used to deliver sanitizing chemicals and solutions.

The bioreactor, if necessary, can be sterilized by filling the bioreactor with sterilizing chemicals (e.g., formaldehyde solution, ethylene oxide, acid or base, ethanol, saturated steam at atmospheric pressure, strong oxidizing agents, or bleach solution) and circulating or otherwise maintaining contact with the bioreactor for a sufficient time to effectively sterilize the unit. Alternatively, the inside of the bioreactor is sterilized by spraying sterilizing chemicals and solutions through the same spray devices and distribution balls 147 used to deliver the sanitizing solutions in step 1. The duration of this step is adjusted to effectively sterilize the inside of the bioreactor.

After sterilization is complete, the bioreactor is filled with sterile air, and then air optionally containing selected gases is continuously pumped in 170 through the sparge tube 130, at a sufficient pressure to maintain a continuous flow of air out of the reactor 150 via the porous material lid 155.

The bioreactor is aseptically filled with an initial growth medium 141 suitable for growth of the production microorganism. The initial growth medium is sterilized prior to adding to the bioreactor and is added via the addition port 165.

The initial growth medium is stirred by the agitator (110, 115, 120, 125) and adjusted to appropriate environmental parameters such as temperature (using external heat exchanger 108), pH (by aseptic addition of acids and bases), nutrient concentration, and dissolved gases, while continuing to pump air optionally containing one or more selected gases into 170 the reactor at a sufficient pressure to maintain a continuous flow through the growth medium and then out of the reactor 150 via the porous material lid 155. Acids, bases, and/or nutrients are added via the addition port 165.

The growth medium is inoculated with a small amount of the production organism supplied by a competent laboratory that meets predetermined quality control specifications. The production organism is added through the addition port 165.

The environmental parameters are monitored and maintained in a range suitable for growth of the production organism. Methods for maintaining environmental parameters are well known to those of ordinary skill in the arts of biochemical engineering, industrial microbiology, and fermentation science, and consist of adjusting air flow through the bioreactor and agitation speed to maintain adequate levels of dissolved gases, circulating a cooling or heating medium through a jacket fitted to the outside wall of the bioreactor to maintain constant temperature, aseptically adding acids, bases and concentrated nutrient solutions to maintain proper pH and levels of nutrients to the growing culture, as examples, depending on the selected cell/organism to be cultured.

During growth of the culture, exhaust gases from the reactor will pass out of the reactor through 150 the porous material cover, lid or dome 155. Exhaust gases typically consist of carbon dioxide and oxygen-depleted air and can be exhausted directly to the atmosphere.

When a sufficient cell mass has been achieved in the bioreactor, the environmental conditions are manipulated by some appropriate means (for example, temperature shift, pH shift, nutrient depletion, begin/stop sugar or carbohydrate feed, add inducer or otherwise shift organism metabolism) to begin production of the primary or secondary metabolite or recombinantly expressed protein. Manipulation of environmental conditions includes continuing to pump gases into the reactor to provide gases for respiration or to facilitate fermentation.

When the producing culture is exhausted or can no longer produce sufficient product of interest, the reactor is drained of biomass, nutrients and other fluids along with the product of interest. Reactor draining occurs through the drain port and drain valve 107.

### Example 2

A Class I bioreactor is designed and assembled that is similar to the bioreactor illustrated in FIGS. 2A-2D with a porous material dome 230 and a porous material filter 262 in the exhaust gas vent 260. The porous material dome 230 is a hemi-spherical/cylindrical shape and the porous material filter 262 is a monolith. Adhesives 285 are used to mount the porous material dome 230 to the vessel wall 205 near the incoming gas duct 275. This bioreactor is utilized for aseptic fermentation for the production of an amino acid. The use of porous material in the bioreactor of this example simultaneously allows sparging of gases into the reactor and prevention of accidental influx of microorganisms that might be present in the gas stream. The use of the porous material improves and extends the bioreactor by combining a sparge device with a hydrophobic sterile barrier. The gas inlet line no longer needs a separate sterilizing filter to exclude contaminating microorganisms. The porous material used as a sparge device cannot backfill with liquid when gas flow stops. The use of porous material in the gas vent line (FIG. 2D) is an incidental example of the use of porous material as a component of a bioreactor.

This bioreactor is employed generally as described in Example 1. During growth of the culture, exhaust gases from the reactor pass out of the vent line. Exhaust gases typically consist of carbon dioxide and oxygen-depleted air and can be exhausted directly to the atmosphere after passing through a sterile barrier material, which is optionally a porous material 262 of this invention. When a sufficient cell mass has been achieved in the bioreactor, the environmental conditions are manipulated by some appropriate means (for example, temperature shift, pH shift, nutrient depletion, begin/stop sugar or carbohydrate feed, add inducer or otherwise shift organism metabolism) to begin production of the amino acid. When the product to be harvested is a constitutively produced product such as an amino acid, nothing other than continued nutrient feed and maintenance of environmental conditions is required. Manipulation of environmental conditions includes continuing to pump gases into the reactor to provide gases for respiration (in the case of amino acid production). When the producing culture is exhausted or can no longer produce sufficient product of interest, the reactor is drained of biomass, nutrients and other fluids along with the product of interest.

### Example 3

A Class II bioreactor is designed and assembled that is similar to the bioreactor illustrated in FIGS. 3A-3B with a porous material cover 360 allowing passage of evolved gas. The porous material is in a flat configuration. This bioreactor is utilized for anaerobic production of hydrogen from starchy algae in the absence of light. The use of porous materials in the exemplary bioreactor of this example improves and extends the bioreactor by providing a single component that simultaneously allows unhindered two-way gas exchange between ambient atmosphere and the culture fluid and excludes foreign microorganisms from the bioreactor.

The bioreactor is cleaned, sterilized and filled with a growth medium as generally described in Example 1 taking into account variations in method required by the different vessel size, shape and geometry and the different process implemented in the bioreactor as would be anticipated by those skilled in the bioreactor art.

An inert gas, e.g., nitrogen or argon that does not interfere with production of the desired product is introduced into the cowl covering the porous material panel of the bioreactor, and the gas is swept through the cowl using the blower. The inert gas flow and cowl negative pressure are controlled so that the inert gas is constantly entering the cowl and gently swept across the face of the bioreactor porous material panel.

The bioreactor aseptically receives a live culture of photosynthetic algae (e.g., a Chlamydomonas culture) that has been photosynthetically cultured elsewhere and has accumulated a sufficient biomass concentration and a sufficient intracellular level of storage polysaccharide to be suitable for hydrogen production under anaerobic conditions. Methods for culturing photosynthetic algae or photosynthetic bacteria at a sufficient scale to provide feed for this bioreactor are known in the art and described elsewhere in this application.

After inoculation the initial growth medium is circulated and environmental parameters such as temperature and pH are maintained, for example (using an external heat exchanger) (and by aseptic addition of acids and bases). Inert gas is continuously introduced into the cowl to reduce the concentration of dissolved oxygen in the culture to zero. Oxygen diffuses out of the culture broth and enters the porous material panel whose open pore structure contains the selected inert gas. Oxygen concentration in the culture fluid falls to zero because the partial pressure of oxygen in the cowl and in the open pore structure is kept infinitely small as inert gas continues to sweep through the cowl.

When the concentration of dissolved oxygen in the culture fluid has reached zero, the inert gas flow into the cowl is stopped, but blower operation is continued so that a slight negative pressure (1 to 5 inches of water column) is maintained in the cowl. Preparations are made to collect gases exiting the exhaust ducting of the blower.

The environmental parameters are monitored and maintained e.g., temperature, pH, hydraulic turbulence and negative cowl pressure in a range suitable for production of hydrogen by the algal culture. Hydrogen is produced, for example, by a Chlamydomonas culture when intracellular storage polysaccharides (carbohydrates) are converted to other more fully oxidized organic compounds such as alcohols, esters, carboxylic acids and ketones. Suitable environmental parameters include zero dissolved oxygen in the culture medium, depletion of a key nutrient under anaerobic conditions, and/or initiating a feed of carbon substrate sufficiently reduced to serve as substrate for the oxidation pathway that produces hydrogen.

The hydrogen produced within the bioreactor is collected. As hydrogen is produced in the culture it exceeds the saturation limit of the broth and appears as small bubbles in the culture fluid. The bubbles migrate through the porous material panel and into the cowl and subsequently enter the suction duct of the blower and are blown out the exhaust ducting into a suitable collection container.

The culture is maintained in a gas-producing state. This involves feeding additional nutrients, harvesting a portion of the cells and returning them temporarily to the photosynthetic conditions that favor accumulation of storage polysaccharides and/or further growth of the culture and then returning them to the subject bioreactor, or using any one of a number of bioprocessing technologies commonly used for fed-batch or continuous culture of live organisms.

When the producing culture is exhausted and can no longer produce the desired quantity of gases, the reactor is drained of all biomass, nutrients and other fluids. The culture may be transferred to another system where photosynthetic conditions favor accumulation of storage polysaccharides and/or further growth of the culture.

### Example 4

A Class II bioreactor is designed and assembled that is similar to the bioreactor illustrated in FIGS. 4A-4B with a transparent porous material monolith window 410 for two-way gas exchange. In this example, there is no forced agitation, and the cell culture requires active respiration. The use of porous materials in the exemplary bioreactor of this invention improves and extends the bioreactor by using a bioreactor wall to simultaneously contain the culture fluid, allow two-way gas exchange with the ambient atmosphere and, if necessary, permit photoradiation to reach the culture fluid. Mass transfer of gas between the ambient atmosphere and the culture fluid is not by molecular diffusion through a solid material or permeable material.

This bioreactor is used only once; it is disposable and not reusable. The reactor is assembled using clean (but not sterile) components under sanitary conditions and is sterilized prior to its use employing sterilizing radiation. The porous material window 410 permits sterilizing short wavelength radiation to illuminate the inside of the reactor during sterilization.

After sterilizing the bioreactor, sterile cell culture media is added aseptically so that the inside surface of the porous material window is in complete contact with the culture fluid. Tipping the reactor by angle theta during filling helps to properly displace the air in the bioreactor so that when full there are no air pockets except in the headspace 420. Note, however, that air inside the reactor is also displaced out of the porous material window during filling. The cell culture fluid can be previously pH balanced and is a complete medium for growth. Additional nutrients or reagents may or may not be required or added.

The bioreactor is placed in an atmosphere/temperature controlled incubator and the temperature of the bioreactor is allowed to equilibrate. The dissolved gas (oxygen and carbon dioxide) concentration in the growth medium is allowed to come to equilibrium with the atmosphere in the incubator by gas exchange through the gas permeable porous material window.

The bioreactor is aseptically inoculated with cells of a plant, insect or mammalian cell line and returned to the controlled temperature incubator.

The culture is allowed to grow and respire. As respiration takes place, O₂ in the medium is depleted and O₂ enters into the culture fluid at the interface between the culture fluid and the air-filled open pore structure of the porous material panel by mass action at the gas-liquid interface. The concentration of O₂ in the growth medium remains at or near equilibrium with the partial pressure of O₂ in the controlled incubator according to Henry's Law. As respiration takes place, the dissolved CO₂ in the medium increases. As respiration continues, CO₂ migrates out of the culture fluid at the interface between the culture fluid and the air-filled open pore structure of the porous material window panel by mass action to maintain the concentration of CO₂ in the growth medium at or near equilibrium with the partial pressure of CO₂ in the incubator. The partial pressure of CO₂ in the incubator, however, is nearly constant and equal to the partial pressure of CO₂ in the atmosphere. If bubbles of CO₂ form in the culture medium, they simply pass through the porous material panel to maintain atmospheric pressure within the bioreactor. Gas samples may be removed from the bioreactor by sampling the headspace using the withdrawal syringe shown in FIGS. 4A and 4B. When used for sampling, the withdrawal syringe is filled sufficiently slowly so that the pressure of the culture fluid in liquid contact with the porous material window is always approximately equal to that of the outside atmosphere.

As cells continue to grow and respire they can anchor to the bottom and sides of the bioreactor as well as to the porous material window. Because the anchored cell mass is low, the additional resistance to gas permeability is low, and the anchored cells do not problematically decrease the two-way gas permeability of the porous material window.

When a sufficient cell mass has been achieved in the bioreactor, the environmental and/or metabolic conditions are manipulated appropriately for the selected cell line (e.g., temperature shift, pH shift, nutrient addition, inducer addition) to begin production of a desired product, e.g., a recombinant protein. Manipulation of environmental and/or metabolic conditions does not reduce or interrupt respiration.

When the producing culture is exhausted or can no longer sufficiently produce the product of interest, the contents of the reactor that are not anchored are harvested using the withdrawal syringe and subjected to further study. The reactor is sterilized in an autoclave to destroy all viable cells and discarded.

### Example 5

A Class III bioreactor is designed and assembled that is similar to the bioreactor illustrated in FIGS. 5A-5C with a porous material lid or cover. The porous material is in a flat configuration. Gasket materials are employed to seal the porous material. This bioreactor is utilized for aseptic fermentation for the production of a pharmaceutical, wherein photoradiation is required for production of the product. The bioreactor illustrated in FIGS. 5A-5C is also useful for batch production of primary and secondary metabolites or engineered proteins using submerged culture fermentation. The use of the porous material improves and extends the bioreactor by simultaneously allowing migration of gases out of the reactor headspace, preventing accidental influx of contaminating organisms or outflow of production microorganisms and, if necessary, permitting illumination of the culture fluid with photoradiation. The porous material eliminates the need for a separate gas vent that also needs to be configured as a sterile barrier.

The bioreactor is cleaned, sterilized, filled with an initial growth medium and inoculated as described in Example 1.

The environmental parameters are monitored and maintained in a range suitable for growth of the production organism. Methods for maintaining environmental parameters are well known to those skilled of biochemical engineering, industrial microbiology, and fermentation science, and consist of optionally providing photoradiation at the required intensity and wavelength, adjusting air flow through the bioreactor and agitation speed to maintain adequate levels of dissolved gases, circulating a cooling or heating medium through a jacket fitted to the outside wall of the bioreactor to maintain constant temperature, aseptically adding acids, bases and concentrated nutrient solutions to maintain proper pH and levels of nutrients to the growing culture, as examples, depending on the selected cell/organism to be cultured.

During growth of the culture, exhaust gases from the reactor will pass out of the reactor through 550 the porous material lid or cover 555. Exhaust gases typically consist of carbon dioxide and oxygen-depleted air and can be exhausted directly to the atmosphere. Photoradiation is received through the porous material cover

Products (primary or secondary metabolites or protein) are produced as described in Example 1. When the producing culture is exhausted or can no longer produce sufficient product of interest, the process is ended and the product is collected as known in the art.

### Example 6

A Class IV bioreactor is designed and assembled that is similar to the bioreactor illustrated in FIGS. 6A-6B with a porous material cover 660, wall, side, or panel. The porous material is two-way gas permeable and photopermeable. Artificial light is optionally delivered by one or more photoradiation sources attached to an optional cowl above the monolith. This bioreactor is used for photosynthetic production of starchy algae. The use of the porous material improves and extends the exemplary bioreactor of this example by incorporating a single component that simultaneously allows unhindered two-way gas exchange between an ambient atmosphere and the culture fluid, excludes foreign microorganisms from the bioreactor and, if necessary, allows illumination of the culture fluid with photoradiation. The porous material puts the culture fluid in direct contact with the ambient atmosphere, while simultaneously maintaining the sterile integrity of the bioreactor contents.

When a cowl 625 is placed over the porous material cover 660 of the bioreactor, air is allowed to enter the space between the cowl and the aerogel panel of the bioreactor through the gas inlets 635.

The unit is cleaned and sanitized as generally described in other Examples. For example, the bioreactor is rinsed with sterile water and sterile air introduced into the piping to fill the reactor with a microbiologically inert fluid. Alternatively, the bioreactor is rinsed with sterile water and air continues to enter the space between the cowl 625 and the porous material cover 660 of the bioreactor and migrate through the porous material cover 660 of the reactor. Alternatively, with no cowl in place, the bioreactor is rinsed with sterile water and air continues to migrate through the porous material cover 660 of the reactor. The air is sterilized as a consequence of moving through the porous material panel because the open pore structure excludes all microorganisms based on size. The inside of the reactor is always at atmospheric pressure.

The bioreactor is aseptically filled with an initial growth medium suitable for growth of the photosynthetic algal culture. The initial growth medium is sterilized prior to being added to the bioreactor. The initial growth medium is circulated and adjusted to appropriate environmental parameters for temperature (using external heat exchanger), pH (by aseptic addition of acids and bases), nutrient concentration and dissolved gases, as appropriate for selected algae. The dissolved gas concentration in the growth medium comes to equilibrium with the atmosphere in the cowl, which is air in this example. The growth medium is innoculated with a small amount of the selected photosynthetic algae supplied by a competent laboratory and meeting predetermined quality control specifications. Alternatively and instead of aseptically filling the bioreactor with an initial growth medium, the bioreactor is filled with a live culture of photosynthetic algae that is returned to photosynthetic conditions for additional growth and/or the accumulation of intracellular storage polysaccharide that can later be converted to, for example, hydrogen gas.

The culture is photoradiated by either energizing a photoradiation source 690 inside the cowl or by allowing sunlight or other artificial lighting into the cowl through photopermeable sections 695 in the cowl. Photopermeable sections 695 of the cowl can comprise porous materials useful in the practice of this invention. When the bioreactor does not have a cowl, the bioreactor porous material cover 660 can be exposed directly to solar radiation or artificial illumination.

The algae is photosynthetically cultured. CO₂ in the medium is depleted as the organism takes up CO₂ and reduces it photosynthetically to organic compounds including storage polysaccharides. As CO₂ in the medium is depleted, CO₂ migrates into the culture fluid at the interface between the culture fluid and the air-filled open pore structure of the porous material cover 660 by mass action, whereby the concentration of CO₂ in the growth medium is at or near equilibrium with the partial pressure of CO₂ in the cowl according to Henry's Law. The partial pressure of CO₂ in the atmosphere within the cowl, however, remains constant or nearly constant as fresh air is allowed to enter the cowl. With no cowl in place, the partial pressure of CO₂ in the atmosphere above the porous material cover 660 remains constant due to the surrounding atmosphere. Fresh air can optionally be sucked into the space under the cowl using a suitable device such as a fan or blower. CO₂ enriched air can optionally be sucked into the space below the cowl using a suitable device such as a fan or blower to elevate the partial pressure of CO₂ in the atmosphere next to the porous material relative to that of the Earth's atmosphere. Suitable sources of air with elevated CO₂ partial pressure include exhaust gases from any combustion source that are suitably clean, i.e., have no toxic byproducts of combustion and no particulate matter that can obscure the transmission of solar or artificial radiation or that otherwise interfere in the culturing of the organism.

The algae continues to be photosynthetically cultured causing dissolved O₂ in the medium to increase. With further photosynthesis, oxygen migrates out of the culture fluid at the interface between the culture fluid and the air-filled open pore structure of the aerogel panel by mass action, maintaining the concentration of O₂ in the growth medium at or near equilibrium with the partial pressure of O₂ under the cowl according to Henry's Law. The partial pressure of O₂ in the atmosphere under the cowl, however, remains constant or nearly constant as fresh air is allowed to enter and move through the space under the cowl. With no cowl in place, the partial pressure of O₂ in the atmosphere above the porous material panel remains constant due to the surrounding atmosphere. Fresh air can be sucked into the space under the cowl using a suitable device such as a fan or blower. O₂-depleted air can also be sucked into the space under the cowl using a suitable device such as a fan or blower to depress the partial pressure of O₂ in the general atmosphere under the cowl, tending to lower the partial pressure relative to that of the Earth's atmosphere. If bubbles of oxygen form in the culture medium, they pass through the porous material panel, maintaining atmospheric pressure within the bioreactor. Bubbles of oxygen mix with the general atmosphere under the cowl, tending to raise the partial pressure of O₂ next to the aerogel. The partial pressure of O₂ in the atmosphere above the aerogel panel, however, remains constant by incoming fresh air, incoming oxygen depleted air or incoming exhaust gases from any combustion source that is suitably clean.

When the photosynthetic culture has reached the desired level of concentration in the medium and/or the desired intracellular level of storage polysaccharide, the reactor is drained of all biomass, nutrients and other fluids. The culture is optionally transferred to another system where conditions favor production and harvest of another product such as hydrogen, e.g., an anaerobic gas harvesting environment.

### Example 7

A Class IV bioreactor is designed and assembled that is similar to the bioreactor illustrated in FIGS. 7A-7B with a porous material cover 760, wall, side, or panel. The cover is sealed with a photopermeable, but gas impermeable seal 762, except for one or more sections covered with a gas harvesting plenum 725. Solar or artificial illumination passes through the photopermeable porous material 760 and seal 762. This bioreactor is utilized to produce hydrogen and CO₂ by photosynthetic bacteria. The use of porous material in the exemplary bioreactor of this example improves and extends the bioreactor by incorporating a single component that simultaneously serves as a structural wall of the bioreactor, permits harvest of gases from a culture fluid, maintains the sterile integrity of the bioreactor contents, and, if necessary allows illumination of the culture fluid with photoradiation. The use of a porous material in the gas vent line illustrates another way to use a porous material as a component of a bioreactor.

The unit is cleaned and sterilized generally as described in the other examples.

The reactor is aseptically filled substantially, but not completely full (allowing a gas headspace 780), with a production medium containing appropriate organic substrates such as organic acids, alcohols, esters, aldehydes, ketones and/or other substrates suitable for the production of hydrogen using a selected cyanobacterium. The production medium, for example, can be taken from spent cultures of photosynthetic algae that have already converted carbohydrates to other more oxidized forms of carbon. During filling, gases within the reactor are displaced through the porous material(s) that constitutes both a bioreactor wall and a standpipe vent.

Environmental parameters such as temperature, pH, and nutrient concentration are appropriately adjusted for the selected cyanobacterium.

The growth medium is inoculated with the production organism, a cyanobacteria culture. The production organism can be supplied by another bioreactor where the cyanobacteria has been previously cultured under aerobic conditions in order to reach a target biomass titer. In order to not substantially dilute the production medium already in the bioreactor, the incoming production organisms can be centrifuged and added as a concentrated suspension or pellet to the subject bioreactor. Volumes of both the production medium and the production organism are adjusted and known in advance so that the subject bioreactor is completely full after the introduction of the production organism.

The production organism begins reducing the dissolved concentration of oxygen to zero as it attempts to continue respiration.

The porous material 760 side of the bioreactor is illuminated with either artificial photoradiation of an appropriate set of wavelengths for the photofermentative production of hydrogen and carbon dioxide by the selected cyanobacteria or by exposing the bioreactor to solar radiation. If necessary, the orientation of the bioreactor is adjusted, using the pivot stand 731, to maximize the incident radiation striking the porous material panel 760, e.g., to achieve incident radiation at 90 degrees to the external planar surface of the porous material.

The blower is turned on to place gas collection plenums under a slight negative pressure and prepare for the collection of gaseous by-products of the photo-fermentation.

When anaerobic conditions have been established in the bioreactor as a natural consequence of respiration, the production organism begins catabolizing the organic substances in the production medium with simultaneous production of hydrogen and CO₂.

Environmental parameters are monitored and maintained, including temperature, pH, natural circulation and negative cowl pressure in a range suitable for maintenance of the cyanobacterial culture. Temperature tends to increase both due to the heat of metabolism as well as due to radiative heating, therefore cooling water is pumped through the internal heating/cooling coil 718 to maintain temperature within a preselected range. Culture pH is adjusted by the addition of small amounts of acid and/or base. Circulation within the bioreactor occurs by natural convection due to small thermal gradients within the reactor.

The gases produced in the bioreactor are collected. Hydrogen and CO₂ are produced, for example, by Rhodovulum and Rhodobacter cultures when organic substrates in the medium (e.g., acids, alcohols, or aldehydes) are fully oxidized to CO₂ under anaerobic conditions and under illumination. As hydrogen and CO₂ are produced in the culture medium they soon exceed the saturation limit of the broth and appear as small bubbles in the culture fluid. The bubbles migrate through the porous material cover 760 and towards the gas collection plenums 725 which are maintained under a slight negative pressure (less than about 1 inch water column) by the blower. CO₂ and hydrogen subsequently enter the suction duct of the blower and are blown out the exhaust ducting into a suitable collection container.

The culture is maintained in a gas-producing state. Suitable environmental parameters required to maintain gas production are specific for the selected organism and biochemical pathway and can include maintaining substantially zero dissolved oxygen in the culture medium and/or initiating a feed of organic substrate for the metabolic pathway that produces CO₂ and hydrogen. If additional liquid is fed to the liquid-filled reactor, some of the contents of the reactor may be displaced into either the standpipe or removed from the bioreactor. Suitable environmental parameters can also include maintaining a maximum level of incident radiation. If the bioreactor is outdoors, the orientation of the bioreactor can be continuously adjusted to track the movement of the sun across the sky. This can be done, for example, by tilting the reactor on the tripod support arrangement shown in the figure. The bioreactor, including the porous material, has been designed and constructed, as is known in the art, to not be structurally or functionally compromised by tilting.

Production of gas products is continued until the producing culture is exhausted and can no longer produce the desired quantity of gas products.

### Example 8

A Class IV bioreactor is designed and assembled that is similar to the bioreactor illustrated in FIGS. 8A-8B using a cylindrical porous material tube 815, to be used with forced circulation and solar illumination. The porous material 815 is sealed to a metal pipe 826 using a compression ferrule 820/821 attachment mechanism. This bioreactor is useful for photosynthetic production of beta-carotene from photosynthetic algae (Dunaliella salina). The use of porous material in the bioreactor of this example improves and extends the bioreactor by simultaneously serving as a structural wall of the bioreactor, putting the culture fluid in direct contact with an ambient atmosphere (the Earth's atmosphere or that of any other composition), preserving the sterile integrity of the culture fluid and allowing illumination of the culture fluid, if desired.

The bioreactor and circulation piping is cleaned, sterilized and filled with a growth medium as generally described in the other Examples and taking into account variations in method required by the different vessel size, shape and geometry and the different process implemented in the bioreactor as would be anticipated by those skilled in the bioreactor art. For example, when the bioreactor is optionally rinsed with clean water air is free to pass through the porous material tube and into the reactor. The air is sterilized as a consequence of moving through the aerogel 815 since the open pore structure excludes all microorganisms based on size. The inside of the reactor will always be at atmospheric pressure. For example, when the bioreactor is filled with a highly salty medium such as would be required for growth of the selected photosynthetic algae, Dunalliela salina, the initial growth medium does not need to be strictly sterilized prior to use since the high salt concentration is unsuitable for the majority of possible contaminating organisms. For example, when the initial growth medium is circulated (circulation piping and pump not shown) and environmental parameters such as temperature (temperature probe and external shell and tube heat exchanger are not shown), pH (pH probe and addition ports for acids and bases are not shown) and nutrient concentration (sample ports and addition ports for nutrients are not shown) are adjusted, the dissolved gas concentrations in the growth medium (both CO₂ and O₂) come to equilibrium with the atmosphere surrounding the tubular reactor, namely air in an outdoor installation.

The growth medium is innoculated with a small amount of the desired photosynthetic algae.

The culture is illuminated by either energizing illumination lights above the parabolic collector surrounding the tubular reactor (tubular reactor sits at the focal point of the paraboloid) or more suitably by orienting the tubular reactor and parabolic cowl to maximize the incident solar radiation in an outdoor installation. The parabolic collector surrounding the tubular reactor can swivel on its focal axis as shown in FIGS. 8A and 8B.

Photosynthetic culture of the organism is continued. In this case, Dunalliela salina depletes CO₂ from the culture medium and reduces it photosynthetically to organic compounds that it uses to synthesize more cell biomass as well as intracellular beta-carotene. As CO₂ in the medium is depleted, CO₂ migrates into the culture fluid at the interface between the culture fluid and the air-filled open pore structure of the porous material cylinder by mass action to maintain the concentration of CO₂ in the growth medium at or near equilibrium with the partial pressure of CO₂ in the atmosphere according to Henry's Law. The partial pressure of CO₂ in the atmosphere, however, remains constant and hence a steady state flux of CO₂ through the aerogel cylinder is established that matches the CO₂ uptake rate of the algal culture. At the same time, oxygen diffuses out of the culture fluid at the interface between the culture fluid and the air-filled open pore structure of the aerogel cylinder by mass action to maintain the concentration of O₂ in the growth medium at equilibrium with the partial pressure of O₂ in the atmosphere which is constant. Bubbles of oxygen can form in the culture medium and pass through the porous material cylinder to maintain constant pressure within the bioreactor.

Optimal conditions for photosynthetic growth are maintained. Methods for maintaining environmental parameters are well known to those skilled in biochemical engineering, industrial microbiology and fermentation science and include maximizing incident solar radiation by adjusting the orientation of the parabolic collector, maintaining a minimum level of mixing in the reactor to ensure minimal temperature and nutrient concentration gradients, maintaining temperature of the culture fluid within a narrow range and aseptically adding acids, bases to maintain proper pH and levels of nutrients to the growing culture. Optimal conditions for photosynthetic growth can also include feeding additional nutrients, harvesting a portion of the cells to prevent the culture fluid from becoming opaque, recovering the biomass and returning the algae-free growth medium to the reactor. These and other bioprocessing techniques are commonly used in the fed-batch, semi-continuous or continuous culture of live organisms and are well known to those skilled in the art of biochemical engineering and industrial microbiology.

When the photosynthetic culture has reached both the desired cell titer and intracellular level of product (beta-carotene, for example) in a batch production protocol OR when the producing culture is exhausted and can no longer produce the desired quantity of either biomass or product, the reactor is drained of all biomass, nutrients and other fluids. The biomass and/or products are recovered by any one of a number of standard biochemical engineering unit operations.

### Example 9

A Class V bioreactor is designed and assembled that is similar to the bioreactor illustrated in FIGS. 9A-9C using porous material in a monolith tent 920 configuration over an open pond for the production of Spirulina blue-green bacteria biomass to be used as a dietary supplement. There are four sides to the open pond, two rectangular sides 910 and two other rectangular or optionally trapezoidal sides 911. The top edges of at least two sides are sealed to the porous material tent cover 920. The use of porous material in the exemplary bioreactor of this example improves and extends the outdoor open-air bioreactor by providing a means for reducing ingress of airborne contaminants (debris, rain, microbes, dust and the like) into the culture fluid, while simultaneously permitting solar or artificial illumination of the culture fluid. If all four sides of the bioreactor are sealed to the porous material tent cover 920 then the use of porous material in this example improves and extends the bioreactor by providing a single component that simultaneously acts as a sterile barrier, permits solar or artificial illumination of the culture fluid and permits two way gas exchange between the Earth's atmosphere and the gas atmosphere immediately above the culture fluid.

The bioreactor is cleaned, sterilized, rinsed and filled with a growth medium as generally described in the other Examples and taking into account variations in method required by the different vessel size, shape and geometry and different processes implemented in the bioreactor as would be anticipated by those skilled in the bioreactor art. During these steps, air freely passes bidirectionally through the porous material cover 920 in the case where all four walls are sealed to the porous material tent cover. The air will be sterilized as a consequence of moving through the porous material cover 920 since the open pore structure excludes all microorganisms based on size. The inside of the reactor is always at atmospheric pressure.

The initial growth medium is circulated either using the recirculation pump and piping 935/930 or using a paddle wheel type agitator 925 mounted inside the bioreactor. Environmental parameters such as temperature (temperature probe and external shell and tube heat exchanger is not shown), pH (pH probe and addition ports for acids and bases are not shown) and nutrient concentration (sample ports and addition ports for nutrients are not shown) are adjusted for the selected organism. The dissolved gas concentrations in the growth medium (both CO₂ and O₂) come to equilibrium with the air under the porous material tent.

The growth medium is inoculated with a small amount of the desired photosynthetic microorganisms. In this example Spirulina algae is cultivated for use as a dietary supplement.

The culture is photoradiated by an artificial photoradiation source (not shown) above the porous material tent 920 or more suitably by installing the bioreactor in a location where it is capable of receiving sunlight.

Photosynthetic culture of the organism proceeds. The active culture of Spirulina consumes CO₂ and evolves O₂. In the case where all four sides are sealed to the porous material tent cover, the porous material tent cover 920 permits gas exchange, and the concentrations of gases under the porous material tent are nearly identical to air. CO₂ passes from the outside air through the porous material tent and to the gas headspace under the porous material tent, and oxygen migrates from the gas headspace under the porous material tent and through the porous material tent to the outside air.

Optimal conditions for photosynthetic growth are maintained as have been generally described in the other Examples and as are well known to those skilled in biochemical engineering, industrial microbiology and fermentation science.

When the photosynthetic culture has reached the desired cell titer in a batch process or can no longer produce the desired quantity of biomass per unit time in a semi-continuous or continuous process the biomass and/or products are recovered by any one of a number of standard biochemical engineering unit operations.

### Example 10

A Class II bioreactor is designed and assembled that is similar to the bioreactor illustrated in FIGS. 4A-4B turned on its side, with a porous material monolith window for two-way gas exchange, wherein the porous window is a portion of a side wall. Sufficient structural support is provided for the porous material. In this example, there is no forced agitation, and the cell culture requires active respiration. The porous window is transparent and optionally photopermeable. The use of porous materials in the bioreactor of this example improves and extends the bioreactor by using a bioreactor wall to simultaneously contain the culture fluid, allow two-way gas exchange with the ambient atmosphere, and, if necessary, permit photoradiation to reach the culture fluid or simply allow visual examination of the culture fluid. The use of a porous material in this example further improves the bioreactor by ensuring its complete disposability and recyclability.

This bioreactor is used only once; it is disposable and not reusable as described for the bioreactor of Example 4. The reactor is assembled and employed as described in Example 4.

### Example 11

A silica aerogel useful in the practice of this invention is made as follows, as adapted from Instruments and Experimental Techniques, vol. 46, No. 3, pp. 287-299:

1) Tetramethooxylane (Si(OCH₃)₄, a silica-containing monomer which is insoluble in water, is dissolved in MeOH.

2) A small amount of NH₄OH is added to catalyze the hydrolysis of tetramethooxylane to orthosilicic acid (Si(OH)₄) and methanol.

3) Orthosilicic acid is unstable, however, and easily polymerized, condensing into silicon dioxide, SiO₂. The polymerization is allowed to spontaneously proceed. Note the polymerization yields the polymer (SiO₂)ₘ as well as 2 m H₂O. Silicon dioxide is produced in the form of spherical colloidal particles about 4 nm in diameter that are linked to each other, forming chains with about 40 nm pores between each particle. The pores are filled with methanol and water. Random end groups have the unreacted hydroxy moiety still attached to Si, which would otherwise give rise to a hydrophilic aerogel, but does not due to step 4 below.

4) A stoichiometric excess amount of hexamethyldisilacene, (CH₃)₃—Si—NH—Si—(CH₃)₃, is added which interacts with the unreacted hydroxyl groups in the SiO₂ polymer and replaces hydrogen in the unreacted OH groups of step 3 with Si(CH₃)₃. A variety of reagents known in the art can be utilized to modify unreacted hydroxyl groups in the SiO₂ polymer making the end groups hydrophobic.

5) The raw polymer obtained in 3) and 4) is allowed to age for approximately one week and solidify within the confines of a mold of a selected shape. The aging polymer is kept under MeOH to promote removal of water from the pores.

6) The aging polymer is dried for approximately 24 hours in an autoclave at a temperature and pressure exceeding those for the critical point for methanol, i.e., critical temp=240° C. and critical pressure=80 atmospheres. Such drying conditions make it possible to avoid the destruction of the porous polymer structure due to the effect of capillary forces. Residual water and methanol are removed from the pores between the particles, and the pores are filled with air, thus forming the aerogel.

### Example 12

A bioreactor tent cover is designed, constructed, and utilized over an open-pond style bioreactor. The bioreactor tent cover is of a photopermeable porous material. The cover is installed over an open pond bioreactor situated in the ground. The cover extends over the edges of the open pond and is supported above the pond, not in direct liquid contact with the medium, allowing air to flow under the cover. Photoradiation from the sun permeates the cover. Rain and possible contaminants do not land in the open pond, but instead fall on the cover. Rain drains off the tent onto the land outside of the pond. The cover is washed periodically by spraying it with water. The cover is of any form that is capable of covering all or part of the pond, optionally a panel or a set of panels. This cover can optionally be part of a bioreactor.

1. A bioreactor comprising a gas-permeable porous material having an open pore structure wherein the porous material is not a support network for a cellular component, cell, or organism to be cultured in the bioreactor, wherein the porous material is a hydrophobic aerogel which is a sterile barrier.

2. The bioreactor of claim 1 wherein the porous material is a portion of a wall, cover, floor, window, or tube of the bioreactor.

3. (canceled)

4. The bioreactor of claim 1 wherein the porous material is a hydrophobic silica aerogel.

5. The bioreactor of claim 1 wherein the hydrophobic aerogel is selectively permeable to one or more of photoradiation, visible radiation, or ultraviolet radiation.

6. The bioreactor of claim 1 wherein the hydrophobic aerogel is selectively gas-permeable.

7. (canceled)

8. The bioreactor of claim 1 wherein the hydrophobic aerogel is photopermeable.

9. The bioreactor of claim 1 wherein the hydrophobic aerogel is transparent.

10-12. (canceled)

13. The bioreactor of claim 1 wherein the hydrophobic aerogel is made by a sol-gel process.

14. (canceled)

15. The bioreactor of claim 1 wherein an entire wall, cover, floor, filter, window, or tube of the bioreactor is made of the hydrophobic aerogel.

16-18. (canceled)

19. The bioreactor of claim 1 wherein a structural element of the bioreactor comprises the hydrophobic aerogel.

20. The bioreactor of claim 1 wherein a structural element of the bioreactor is made of the hydrophobic aerogel.

21. The bioreactor of claim 1 also comprising one or more structural elements, functional elements or both which alone or in combination provide a means for:
sanitization and/or sterilization;
contamination prevention;
monoseptic holding or processing;
culture agitation or circulation;
temperature detection, temperature control, or both;
gas delivery, gas removal, or both removal;
dissolved gas detection, gas control, or both
pH detection, pH control, or both;
photoradiation delivery;
detecting photoradiation; quantitating photoradiation, or both;
reorientation of a portion of the bioreactor relative to a source of photoradiation;
liquid delivery, liquid removal, or both;
nutrient detection, nutrient delivery or both;
waste removal;
cell and organism delivery, cell and organism removal or both;
gas harvesting; or
product harvesting.

22. The bioreactor of claim 1 wherein the hydrophobic aerogel has a pore size between 1 and 500 nanometers.

23-35. (canceled)

36. A method for producing a product selected from the group consisting of gaseous products, biomass, chemicals, and pharmaceuticals, the method comprising:
(a) providing a bioreactor of claim 1;
(b) providing a cell, organism, or cellular component capable of producing the product;
(c) providing environmental conditions whereby the cell, organism, or cellular component produces the product; and
(d) collecting the product.

37. The bioreactor of claim 1 wherein the hydrophobic aerogel is in the form of a tube and in combination with recirculation piping forms a vessel for circulation of culture fluid.

38. The bioreactor of claim 37 wherein the hydrophobic aerogel is photopermeable and the bioreactor further comprises a parabolic photoradiation collector and wherein the hydrophobic aerogel tube of the vessel for circulation of culture fluid is positioned along the focal axis of the parabolic photoradiation collector.

39. A bioreactor comprising a culture chamber wherein a hydrophobic aerogel is a portion of a wall, panel, cover, floor, window, or tube of the bioreactor in fluid communication with the gas within the culture chamber and wherein the bioreactor further comprises a cowl or plenum external to the culture chamber and in fluid communication with the hydrophobic aerogel, such that gas is exchanged between the culture chamber and the cowl or plenum through the hydrophobic aerogel, wherein the hydrophobic aerogel provides a sterile barrier.

40. The bioreactor of claim 39 which comprises a cowl, wherein the cowl is operationally connected to a fan, blower or pump to discharge and simultaneously refresh the gas atmosphere in the cowl.

41. The bioreactor of claim 40 wherein at least a portion of the cowl is photopermeable and wherein the hydrophobic aerogel is photopermeable.

42. The bioreactor of claim 40 wherein the hydrophobic aerogel is photopermeable and wherein one or more artificial sources of photoradiation are provided in the cowl to provide photoradiation which passes through the hydrophobic aerogel into the culture chamber.

43. The bioreactor of claim 40 wherein the cowl is supplied with an inert gas.

44. The bioreactor of claim 39 wherein the bioreactor comprises a plenum operationally connected to a blower for pulling gases from the plenum for collection.

45. The bioreactor of claim 44 wherein the outer surface of the hydrophobic aerogel that is not in contact with the plenum is sealed with a photopermeable gas impermeable film.

46. The bioreactor of claim 44 wherein the hydrophobic aerogel is photopermeable.

47. The bioreactor of claim 46 wherein the bioreactor is operationally connected to a pivot axis and pivot stand for tilting the bioreactor to maximize receipt of photoradiation.

48. A method for producing a product selected from the group consisting of gaseous products, biomass, chemicals, and pharmaceuticals, the method comprising:
(e) providing a bioreactor of claim 39;
(f) providing a cell, organism, or cellular component capable of producing the product;
(g) providing environmental conditions whereby the cell, organism, or cellular component produces the product; and
(h) collecting the product.

49. A bioreactor comprising:
a body that functions as a vessel for containing medium which has an opening therein and an elevated portion thereof, a monolith window which serves as a cover for the opening in the body, the elevated portion of the body extending above the opening to provide a gas headspace in a portion of the vessel, wherein medium in the bioreactor is at a level equal to or higher than the opening in the body such that the medium in the bioreactor is in contact with the entire monolith window;
wherein the monolith window is made of hydrophobic aerogel that provides a sterile barrier.

50. The bioreactor of claim 49 further comprising septum ports for syringes.

51. The bioreactor of claim 50 wherein a first septum port is positioned in the elevated portion of the body for withdrawing gas from the gas headspace and a second septum port is positioned in the body for making liquid additions to the vessel.

52. The bioreactor of claim 49 wherein the hydrophobic aerogel is photopermeable.

53. A method for producing a product selected from the group consisting of gaseous products, biomass, chemicals, and pharmaceuticals, the method comprising:
(i) providing a bioreactor of claim 49;
(j) providing a cell, organism, or cellular component capable of producing the product;
(k) providing environmental conditions whereby the cell, organism, or cellular component produces the product; and
(l) collecting the product.
